SEQUENCE LISTING (4 sequences)

SEQ ID NO: 1 - protein, 193 aa, Homo sapiens
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 2 - protein, 313 aa, Homo sapiens
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 3 - DNA, 21 bp, Artificial Sequence
  GAPDH primers
ccaggttgtctcctgcgactt

SEQ ID NO: 4 - DNA, 22 bp, Artificial Sequence
  GAPDH primer
cctgttgctgtagccgtattca

(19) United States
(12) Patent Application Publication
(10) Pub. No.: US 2020/0147177 A1
(43) Pub. Date: May 14, 2020

(54) INTERLEUKIN 12 (IL12) OR DERIVATIVE THEREOF FOR USE IN THE TREATMENT OF SECONDARY DISEASE

(71) Applicants: CHU DE NANTES - CENTRE HOSPITALIER UNIVERSITAIRE DE NANTES, NANTES (FR); UNIVERSITE DE NANTES, NANTES (FR); THE UNIVERSITY OF MELBOURNE, VICTORIA (AU)

(72) Inventors: ANTOINE ROQUILLY, NANTES (FR); KARIM ASEHNOUNE, NANTES (FR); JOSE VILLADANGOS, MELBOURNE (AU)

(21) Appl. No.: 16/631,687

(22) PCT Filed: Jul. 16, 2018
(86) PCT No.: PCT/EP2018/069190

§ 371 Date: Jan. 16, 2020

(30) Foreign Application Priority Data
Jul. 17, 2017 (EP) 17305945.2
Nov. 10, 2017 (EP) 17201074.6

(51) Int. Cl. A61K 38/20; A61P 31/04

(52) U.S. Cl. CPC A61K 38/208; A61P 31/04

(57) ABSTRACT

The invention relates to Interleukin 12 (IL12) or derivative thereof for use in the treatment of secondary infection. The present invention also relates to pharmaceutical composition comprising Interleukin 12 (IL12) or derivative thereof for use in the treatment of secondary infection. The present invention finds application in the therapeutic and diagnostic medical technical fields.

### FIELD OF THE INVENTION

The invention relates to Interleukin 12 (IL12) or derivative thereof for use in the prevention and/or the treatment of secondary disease, in particular nosocomial disease.

The present invention also relates to pharmaceutical composition comprising Interleukin 12 (IL12) or derivative for use in the prevention and/or the treatment of secondary disease, in particular nosocomial disease.

The present invention finds application in the therapeutic and diagnostic medical technical fields.

### BACKGROUND OF THE INVENTION

Pneumonia is the leading cause of death from infectious disease (Mizgerd, 2006 [41]). The risk of developing pneumonia increases following severe primary infections and reaches 30-50% for critically ill patients recovering from a first episode of infection (van Vught et al., 2016a [59]). It is currently accepted that susceptibility to secondary pneumonia increases due to acquired immune defects collectively known as sepsis-induced immunosuppression (Hotchkiss et al., 2013a [26]; Roquilly and Villadangos, 2015 [49]). In-depth understanding of the mechanisms involved is vital to prevent and treat secondary pneumonia in patients recovering from a primary infection.

Healthy lungs are colonized by bacteria whose burden is continuously controlled by mucosal immunity (Charlson et al., 2011 [16]). Infection by pathogenic bacteria disrupts this balance and can induce lung injury through direct damage caused by the pathogen, or through immunopathology elicited by the effector mechanisms of immunity. Therefore, a healthy immune response should maximize the deployment of effector mechanisms against the pathogen while minimizing the damage of self-tissues that may ensue.

It is well known that Nosocomial infections (NI) increase morbidity and mortality. In particular, the most common NI are surgical site infections, infections of the gastrointestinal tract and respiratory tract, urinary tract infections, and primary sepsis. (Ella Ott, Dr. med., et al. “The Prevalence of Nosocomial and Community Acquired Infections in a University Hospital An Observational Study Dtsch Arztebl Int. 2013 August; 110(31-32): 533-540 [69]).

In addition, it is well known that the NI, when due to bacterial infection, are strong infection which are, in most of the time, resistant to the most common antibiotic compound. Thus, these therapies have to be improved since they do not allow to effectively treat the NI and/or are less effective in the treatment than expected.

There is therefore a real need to find a method and/or a compound which allows more efficient treatment and/or effective treatment of Nosocomial Infections (NI). In particular there is a real need to find new strategies, i.e. new targets/pathways, in the treatment Nosocomial infections (NI).

### DESCRIPTION OF THE INVENTION

The present invention meets these needs and overcomes the abovementioned drawbacks of the prior art with the use of Interleukin 12 for the prevention and/or treatment of secondary disease, in particular nosocomial disease.

In particular, the macrophages and dendritic cells (DC) orchestrate immunity and tolerance, the inventors have compared their functional properties before, during and after resolution of a first infection, for example pneumonia and demonstrated that both cell types showed profound alterations—which we summarize as ‘paralysis’—in the latter case. Paralysis was caused by excessive release of local mediators of restoration of immune homeostasis. The inventors have supported that DC and macrophage dysfunction is an important contributor to protracted immunosuppression after bacterial or viral primary sepsis and increased susceptibility to secondary infection, for example Nosocomial Infections (NI) such as a secondary pneumonia.

The inventors have also demonstrated that the use of Interleukin 12 allows to treat secondary infection, for example Nosocomial infections whatever is the Nosocomial infection. In other words, the inventors have demonstrated that Interleukin 12 allows to treat Nosocomial infections and also to inhibit protracted immunosuppression after, for example bacterial and/or viral and/or fungus primary sepsis and/or infections.

The inventors have also demonstrated that the use of inhibitor of transforming growth factor-beta allows to treat secondary infection, for example Nosocomial infections whatever is the secondary infection, for example Nosocomial infection. In other words, the inventors have also demonstrated that the use of inhibitor of transforming growth factor-beta inhibitor allows to treat secondary infections, for example Nosocomial infections and also to inhibit the protracted immunosuppression after bacterial and/or viral and/or fungus primary sepsis.

The inventors have also demonstrated that Dendritic Cells (DC), for example DC that develop in the lung after resolution of a first infection, for example pneumonia have diminished capacity to present antigens and to secrete immunostimulatory cytokines, which makes them less capable of initiating adaptive and innate immunity against a secondary infection, for example a bacterial and/or viral and/or fungus infection. In addition, they produce higher levels of TGF-β, which promotes accumulation of Treg cells.

The inventors have also demonstrated that the signals that promote the differentiation of DC to this paralyzed state are not directly associated with the pathogen that caused the primary infection; they are mediated by secondary cytokines acting locally. Accordingly, this effect appears whether the disease and/or the pathogen. Accordingly, the inventors have demonstrated that IL-12 or inhibitor of transforming growth factor-beta allow to treat any secondary infection, for example nosocomial infection.

The inventors have also demonstrated that Interleukin 12 allows to treat secondary infections, nosocomial infections and/or also to inhibit protracted immunosuppression after, for example bacterial and/or viral and/or fungus primary sepsis and/or infections and/or after conditions that could induce primary inflammation, for example trauma, hemorrhage, infection.

In addition, the inventors have also demonstrated that Interleukin 12 (IL-12) allows to prevent secondary infections in a systemic way. In other words, the inventors have demonstrated that after a primary condition, for example bacterial and/or viral and/or fungus primary sepsis and/or infections and/or after conditions that could induce primary inflammation, for example trauma, hemorrhage and/or infection; IL-12 allows to prevent secondary infection whatever the localization and/or the organ infected. In particular, the inventors have demonstrated unexpectedly that IL-12 provides a systemic protection which advantageously would allow to prevent and/or treat a secondary infection which could appear at different localization and/or in different organ with regards to the primary infection.

Moreover, the inventors have demonstrated that the present invention allows surprisingly and unexpectedly to prevent and/or treat secondary infections whatever the cause of the secondary infections. In other words, the origin and/or cause of the secondary infection may be advantageously different from the origin and/or cause of the primary infection.

An object of the present invention is Interleukin 12 (IL12) or derivative thereof for use in the prevention and/or the treatment of secondary infection.

Another object of the invention is interleukin 12 (IL12) or derivative thereof for use as a medicament in the prevention and/or the treatment secondary infection.

In the present interleukin 12 refers to a heterodimeric cytokine encoded by two separate genes, IL-12A (p35) and IL-12B (p40).

In the present interleukin 12 may be any interleukin 12 known from one skilled in the art that can be administered to a patient in need thereof. It may be for example a commercially available Interleukin 12, for example Interleukin 12 commercialized by Abcam, a recombinant human interleukin 12 (rHuIL-12) as disclosed in Gokhale et al. Single low dose rHuIL-12 safely triggers mutyilineage hematopoietic and immune-mediated effects, Experimental Hematology & oncology 2014, 3:11, pages 1-18 [70].

In the present interleukin 12 may be heterodimeric cytokine comprising an IL-12A (p35) amino acid sequence of RVIPVSGPARCLSQSRNLLKTTDDMVKTAREKLKHYSCTAEDIDHEDITRDQT STLKTCLPLELHKNESCLATRETSSTTRGSCLPPQKTSLMMTLCLGSIYEDLK MYQTEFQAINAALQNHNHQQIILDKGMLVAIDELMQSLNHNGETLRQKPPVG EADPYRVKMKLCILLHAFSTRWTINRVMGYLSSA (SEQ ID NO 1) and an IL-12A (p40) amino acid sequence of MWELEKDVYVVEVDWTPDAPGETVNLTCDTPEEDDITWTSDQRHGVIGSGK TLTITVKEFLDAGQYTCHKGGETLSHSHLLLHKKENGIWSTEILKNFKNKTFLK CEAPNYSGRFTCSWLVQRNMDLKFNIKSSSSSPDSRAVTCGMASLSAEKVT LDQRDYEKYSVSCQEDVTCPTAEETLPIELALEARQQNKYENYSTSFFIRDIIK PDPPKNLQMKPLKNSQVEVSWEYPDSWSTPHSYFSLKFFVRIQRKKEKMKE TEEGCNQKGAFLVEKTSTEVQCKGGNVCVQAQDRYYN SSCSKWACVPCRVRS (SEQ ID NO 2).

In the present, derivative of IL12 may any derivative of IL-12 known to one skilled in the art. For example, derivative of IL12 may be acetylated IL12, for example an acetylated, alkylated, methylated, methylthiolated, biotinylated, glutamylated, glycylated, glycosylated, hydroxylated, isoprenylated, prenylated, myristoylated, farnesylated, geranyl-geranylated, lipoylated, Phosphopantetheinylated, phosphorylated, sulphated, selenated or amidated IL-12.

For example acetylation may be carried out with addition of an acetyl group derived from acetyl-CoA at the N-terminal end; alkylation, or the addition of an alkyl, methyl or ethyl group; methylation may be carried out with addition of a methyl group, generally on the amino acids lysine or arginine; methylthiolation may be carried out with addition of a methylthio group; biotinylation may be carried out with the acylation of a lysine by a biotin group; glutamylation may be carried out with covalent bonding of a glutamic acid residue to tubulin or other protein; glycylation, may be carried out with covalent bond of one or more (up to 40) glycine residues to the C-terminal end; Glycosylation may be carried out with addition of a glycosyl group to an asparagine, hydroxylysine, serine, or threonine residue, hydroxylation, may be carried out with addition of a hydroxyl group to a protein, most often on a proline or lysine residue forming hydroxyproline or hydroxylysine; isoprenylation, may be carried out with addition of an isoprenoid group, for example farnesol or geranylgeraniol; phosphopantetheinylation may be carried out with addition of a 4′-phosphopantetheinyl from coenzyme A, phosphorylation, may be carried out with addition of a phosphate group, typically on an acceptor serine, tyrosine, threonine or histidine; sulphation may be carried out with addition of a sulfate group to a tyrosine.

In the present derivative of IL12 may also encompass pro-drug of IL-12. In the present, pro-drug of IL-12 may any pro-drug of IL-12 known to one skilled in the art. For example prodrug of IL12 may be IL-12 modified with polymers, for example IL-12 conjugated with polyethylene glycol (PEG), IL-12 conjugated with polyoxyethylated glycerol, with polymers.

Interleukin 12 (IL12) or derivative thereof can be administered to humans and other animals orally, rectally, parenterally, intratracheally, intracisternally, intravaginally, intraperitoneally, topically (as by powders, ointments or drops), buccally, as oral or nasal spray, subcutaneously, or the like, depending on the severity of the infection to be treated. For example, the interleukin 12 (IL12) or derivative thereof may be administered, for example subcutaneous at doses of from about 2 to 20 μg, preferably from 5 to 15 μg, preferably equal to 12.5 μg on a single bolus.

For example, the interleukin 12 (IL12) or derivative thereof may be administered, for example subcutaneous at doses of from about 0.1 μg/kg to 1 μg/Kg body weight of the subject per day, one or more times a day, to achieve the desired therapeutic effect.

According to the invention, interleukin 12 (IL12) or derivative thereof may be administered on a single administration or repeated administration, for example one to three time per day, for example for a period up to 21 days.

In the present inhibitors of transforming growth factor-beta (TGF-β) may be any inhibitors known from one skilled in the art. It may be for example comprising antibodies against transforming growth factor-beta, antisense oligo, peptides, mouse antibody, ligand trap, small molecules, pyrrole-imidazole polyamide, inhibitor or TGF-β synthesis, humanized antibody.

In the present antibodies against transforming growth factor-beta may be any corresponding antibody known from one skilled in the art. It may be for example a commercially available antibodies. It may be for example antibody of any mammal origin adapted for the treatment of human being. It may be for example, antibodies obtained according to the process disclosed in Leffleur et al. 2012 [37] comprising administering 0.3 to 8 mg/kg of anti-tgf beta antibody (Trachtman et al. 2012 [55]).

In the present antibodies against transforming growth factor-beta may be mouse antibody, for example any mouse antibody known from one skilled in the art that could inhibit transforming growth factor-beta. It may be for example a commercially available mouse antibody, for example mouse antibody referenced 1D11, 2AR2, X1, 2C6, 8C4.

In the present antibodies against transforming growth factor-beta may be mouse antibody, for example any rat antibody known from one skilled in the art that could inhibit transforming growth factor-beta. It may be for example a commercially available rat antibody, for example rat antibody referenced TB2F.

In the present antibodies against transforming growth factor-beta may be a rabbit antibody, for example any rabbit antibody known from one skilled in the art that could inhibit transforming growth factor-beta. It may be for example a commercially available rabbit antibody, for example rabbit antibody referenced ab92486 commercialized by abcam or aa279)-390 commercialized by antibodies-online.com.

In the present antisense oligo may be any corresponding antisense oligo known from one skilled in the art that could inhibit transforming growth factor-beta. It may be for example a commercially available antisense oligo, for example P144, P17, LSKL commercialized by Trabedersen, Belagen-pumatucel-L.

In the present peptide may be any peptide known from one skilled in the art that could inhibit transforming growth factor-beta. It may be for example a commercially available peptides, for example peptide referenced P144, P17 or LSKL.

In the present ligand trap may be any ligand trap known from one skilled in the art that could inhibit transforming growth factor-beta. It may be for example a commercially available ligand trap, for example ligand trap referenced SR2F and/or soluble TbR2-Fc.

In the present small molecules may be any small molecules known from one skilled in the art that could inhibit transforming growth factor-beta. It may be for example a commercially available small molecules, for example small molecules referenced LY580276, LY550410, LY364947, LY2109761, SB-505124, SB-431542, SD208, SD093, Ki26894, SM16 and/or GW788388.

In the present pyrrole-imidazole polyamide may be any pyrrole-imidazole polyamide known from one skilled in the art that could inhibit transforming growth factor-beta. It may be for example a commercially available pyrrole-imidazole polyamide, for example pyrrole-imidazole polyamide referenced GB1201, GB1203.

In the present inhibitor of TGFb synthesis may be inhibitor of TGFb synthesis known from one skilled in the art. It may be for example a commercially available inhibitor of TGFb synthesis, for inhibitor of TGFb synthesis referenced (Lucanix), a humanized antibody, for example a commercially available humanized antibody selected from the group comprising Lerdelimumab (CAT-152) Metelimumab (CAT-192) Fresolimumab (GC-1008), LY2382770; STX-100, IMC-TR1).

In the present inhibitor of TGFb may also be any inhibitor disclosed in Akhurst et al, targeting the TGFβ signaling pathway in disease, Nature reviews, drug discovery Vol 11, October 2012, p 790-812 [1].

Inhibitors of transforming growth factor-beta (TGF-β) may be administered to humans and other animals orally, rectally, parenterally, intracisternally, intravaginally, intraperitoneally, topically (as by powders, ointments or drops), buccally, as oral or nasal spray, subcutaneously, or the like, depending on the severity of the infection to be treated.

The way of administration of inhibitors of transforming growth factor-beta (TGF-β) may be adapted with regards to the inhibitor used. One skilled in the art taking into consideration his technical knowledge would adapt the administration way to the used inhibitor.

The doses of inhibitors of transforming growth factor-beta (TGF-β) to be administered may be adapted with regards to the inhibitor used. One skilled in the art taking into consideration his technical knowledge would adapt the administered doses to the used inhibitor. For example, when the inhibitors of transforming growth factor-beta (TGF-β) is small molecules, for example LY2157299, it may be administered, for example at doses around 80 mg. For example, when the inhibitors of transforming growth factor-beta (TGF-β) is recombinant protein, for example Avotermin, it may be administered, for example at doses from 20 ng to 200 ng, preferably from 50 ng to 200 ng, more preferably 100 ng to 200 ng. For example, when the inhibitors of transforming growth factor-beta (TGF-β) is humanized antibody, for example IMC-TR1, it may be administered, for example at doses from 12.5 mg to 1600 mg.

According to the invention, inhibitors of transforming growth factor-beta (TGF-β) may be administered on a single time or repeated administration, for example one to three time per day, for example for a period up to 21 days.

In the present secondary infection means any infection which may occur after a primary infection and/or inflammation and/or postoperatively. It may be for example an infection occurring 1 to 28 days after the beginning of a primary infection, for example 5 to 12 day after the beginning of a primary infection. It may be also for example an infection occurring 1 to 21 days after the end of a primary infection for example 5 to 12 day after the end of the primary infection and/or the absence of any pathological sign and/or symptom.

In the present the secondary infection may be for example the origin and/or cause of the secondary infection may be advantageously different from the origin and/or cause of the primary infection.

In the present the secondary infection may for example affect other organ or another part of the subject compares to the primary infection, and/or inflammation. In other words, the secondary infection may affect an organ and/or part of the body which is different from the organ and/or part of the body infected by the primary infection and/or inflammation.

In the present the secondary infection may be any infection occurring after a primary infection known to one skilled in the art. It may be for example any secondary infection of gastrointestinal tract, respiratory tract, urinary tract infections. It may be for example any secondary infection of organ selected for the group comprising lung, liver, eye, heart, breast, bone, bone marrow, brain, mouth, head & neck, esophageal, tracheal, stomach, colon, pancreatic, cervical, uterine, bladder, prostate, testicular, skin, rectal, and lymphomas.

In the present the secondary infection may be a secondary infection selected from the group comprising pneumonia, pleural infection, urinary infection, peritoneal infection, intra-abdominal abscess, meningitis, mediastinal infection, soft-tissue or skin infection, such as cellulitis). For example it may be a secondary infection selected from the group comprising pneumonia, pleural infection, urinary infection, peritoneal infection, intra-abdominal abscess, meningitis and mediastinal infection.

In the present, the secondary infection may be due to any pathogen known to one skilled in the art. The secondary infection may be due to a bacteria selected from the group comprising Staphylococcus aureus, Methicillin resistant Staphylococcus aureus, Streptococcus pneumonias, Pseudomonas aeruginosa, Enterobacter spp (including E. cloacae), Acinetobacter baumannii, Citrobacter spp (including C. freundii, C. koserii), Klebsiella spp (including K. oxytoca, K. pneumoniae), Stenotrophomonas maltophilia, Clostridium difficile, Escherichia coli, Heamophilus influenza, Tuberculosis, Vancomycin-resistant Enterococcus, Legionella pneumophila. Other types include L. longbeachae, L. feeleii, L. micdadei, and L. anisa.

In the present, the secondary infection may be due to any virus known to one skilled in the art. It may be for example any virus mentioned in CELIA AITKEN et al. “Nosocomial Spread of Viral Disease” Clin Microbiol Rev. 2001 July; 14(3): 528-546 [15]. It may be due to a virus selected from the group comprising RSV, influenza viruses A and B, parainfluenza viruses 1 to 3, rhinoviruses, adenoviruses, measles virus, mumps virus, rubella virus, parvovirus B19, rotavirus, enterovirus, hepatitis A virus, hepatitis B virus, hepatitis C virus, herpes simplex virus (HSV) types 1 and 2, Varicella-Zoster Virus (VZV), Cytomegalovirus (CMV), Epstein Barr virus (EBV), and human herpesviruses (HHVs) 6, 7, and 8, Ebola virus, Marburg virus, Lassa fever virus, Congo Crimean hemorrhagic fever virus, Rabies virus, Polyomavirus (BK virus).

In the present, the secondary infection may be due to any fungus known to one skilled in the art. It may be for example any fungus disclosed in SCOTT K. FRIDKIN et al. “Epidemiology of Nosocomial Fungal Infection” Clin Microbiol Rev, 1996; 9(4): 499-511 [51]. The secondary infection may be due to a specie of fungus selected from the group comprising Candida spp, Aspergillus spp, Mucor, Adsidia, Rhizopus, Malassezia, Trichosporon, Fusarium spp, Acremonium, Paecilomyces, Pseudallescheria.

In the present the secondary infection may be a nosocomial infection. It may be a nosocomial infection of any organ as previously mentioned. It may be a nosocomial infection due to any pathogen selected from the group comprising virus, bacteria and fungus. It may be a nosocomial infection due to a virus as previously defined. It may be a nosocomial infection due to a bacteria as previously defined. It may be a nosocomial infection due to a fungus as previously defined. It may be nosocomial infection selected from the group comprising pneumonia, pleural infection, urinary infection, peritoneal infection, intra-abdominal abscess, meningitis, mediastinal infection. It may be nosocomial infection selected from the group comprising pneumonia, pleural infection, urinary infection, peritoneal infection, intra-abdominal abscess, meningitis, mediastinal infection, soft-tissue or skin infection (cellulitis), head & neck infection (including otitis).

The secondary infection may be a nosocomial infection, in particular pneumonia

The secondary infection may be a nosocomial infection, for example an infection originated from hospital and/or acquired at the hospital and/or hospital-acquired infection.

In a particular embodiment, the secondary infection may be secondary pneumonia and/or a hospital-acquired pneumonia.

In the present primary infection means an infection due to any pathogen, or sepsis-like syndrome, that could have a negative effect on immune response and/or induce an immunosuppression

In the present primary infection means an infection due to a pathogen selected from the group comprising bacteria, virus or fungus. It may be for example any infection due to pathogen selected from the group comprising bacteria, virus or fungus known from one skilled in the art. It may be for example an infection of gastrointestinal tract, respiratory tract, urinary tract infections, and primary sepsis. It may be for example any infection due to a pathogen selected from the group comprising virus, bacteria and fungus. It may be for example a non-documented infection, for example an infection wherein no pathogens have been searched or found, such as sepsis-like syndrome. In the present the primary infection may be any infection due to a pathogen of at least one organ selected for the group comprising lung, liver, eye, heart, breast, bone, bone marrow, brain, head and neck, esophageal, tracheal, stomach, colon, pancreatic, cervical, uterine, bladder, prostate, testicular, skin, rectal, and lymphomas.

Another object of the present invention is a pharmaceutical composition comprising interleukin 12 (IL12) or derivative thereof and a pharmaceutically acceptable carrier.

The Interleukin 12 (IL12) or derivative thereof is as defined above.

Another object of the present invention is a pharmaceutical composition comprising inhibitor of transforming growth factor-beta and a pharmaceutically acceptable carrier.

The inhibitor transforming growth factor-beta is as defined above.

The pharmaceutical composition may be in any form that can be administered to a human or an animal. The person skilled in the art clearly understands that the term “form” as used herein refers to the pharmaceutical formulation of the medicament for its practical use. For example, the medicament may be in a form selected from the group comprising an injectable form, aerosols forms, an oral suspension, a pellet, a powder, granules or topical form, for example cream, lotion, collyrium, sprayable composition.

As described above, the pharmaceutically acceptable compositions of the present invention further comprise a pharmaceutically acceptable carrier, adjuvant or carrier. The pharmaceutically acceptable carrier may be any known pharmaceutical support used for the administration to a human or animal, depending on the subject to be treated. It may be any solvent, diluent or other liquid carrier, dispersion or suspension, surfactant, isotonic agent, thickening or emulsifying agent, preservative, solid binder, lubricant and the like, adapted to the particular desired dosage form. Remington Pharmaceutical Sciences, sixteenth edition, E. W. Martin (Mack Publishing Co., Easton, Pa., 1980) discloses various carriers used in the formulation of pharmaceutically acceptable compositions and known techniques for their preparation. Except in the case where a conventional carrier medium proves incompatible with the compounds according to the invention, for example by producing any undesirable biological effect or by deleteriously interacting with any other component of the pharmaceutically acceptable composition, Its use is contemplated as falling within the scope of the present invention. Some examples of materials which can serve as pharmaceutically acceptable carriers include, but are not limited to, ion exchangers, alumina, aluminum stearate, lecithin, serum proteins such as human serum albumin, Buffer substances such as phosphates, glycine, sorbic acid or potassium sorbate, mixtures of partial glycerides of saturated vegetable fatty acids, water, salts or electrolytes such as protamine sulphate, Disodium phosphate, potassium hydrogen phosphate, sodium chloride, zinc salts, colloidal silica, magnesium trisilicate, polyvinylpyrrolidone, polyacrylates, waxes, polyethylene-polyoxypropylene polymers, sugars such as lactose, Glucose and sucrose; Starches such as corn and potato starch; Cellulose and derivatives thereof such as sodium carboxymethylcellulose, ethylcellulose and cellulose acetate; Tragacanth powder; Malt; Gelatin; Talc; Excipients such as cocoa butter and suppository waxes; Oils such as peanut oil, cottonseed oil; Safflower oil; Sesame oil; olive oil; Corn oil and soybean oil; Glycols; Such a propylene glycol or polyethylene glycol; Esters such as ethyl oleate and ethyl laurate; Agar; Agents such as magnesium hydroxide and buffered aluminum hydroxide; Alginic acid; Isotonic saline; Ringer's solution; Ethyl alcohol, and phosphate buffer solutions, as well as other compatible non-toxic lubricants such as sodium lauryl sulfate and magnesium stearate, as well as coloring agents, release agents, coating agents, sweetening, flavoring and perfuming agents, preservatives and antioxidants may also be present in the composition, according to the judgment of the galenist.

The pharmaceutical form or method of administering a pharmaceutical composition may be selected with regard to the human or animal subject to be treated. For example it may be administered to humans and other animals orally, rectally, parenterally, intratracheally, intracisternally, intravaginally, intraperitoneally, topically (as by powders, ointments or drops), buccally, as oral or nasal spray, subcutaneously, or the like, depending on the severity of the infection to be treated. The pharmaceutical form or method of administering a pharmaceutical composition may be selected with regard to the site of infection and/or infected organ. For example, for an infection of the respiratory tract it may in a form adapted to be administered to humans and other animals as oral or nasal spray or parenteral or intratracheal, for an infection of the gastrointestinal tract it may in a form adapted to be administered to humans and other animals orally, for example a pellet, a capsule, a powder, granules, a syrup or parenteral or intraperitoneal. The pharmaceutical form or method of administering a pharmaceutical composition may be selected with regard to the age of the human to be treated, and/or with regard to comorbidity, associated therapies and/or site of infection. For example, for a child, for example from 1 to 17 years old, or a baby, for example under 1 year old, a syrup or an injection, for example subcutaneous or intravenous may be preferred. Administration may for example be carried out with a weight graduated pipette, a syringe. For example, for an adult over 17 years old, an injection may be preferred. Administration may be carried out with an intravenous weight graduated syringe.

According to the present invention, the pharmaceutical composition may comprise any pharmaceutically acceptable and effective amount of interleukin 12 (IL12) or derivative thereof.

According to the present invention, the pharmaceutical composition may comprise any pharmaceutically acceptable and effective amount of inhibitor of transforming growth factor-beta.

In this document, an “effective amount” of a pharmaceutically acceptable compound or composition according to the invention refers to an amount effective to treat or reduce the severity of nosocomial disease. The compounds and compositions according to the method of treatment of the present invention may be administered using any amount and any route of administration effective to treat or reduce the severity of a nosocomial disease or condition associated with. The exact amount required will vary from one subject to another, depending on the species, age and general condition of the subject, the severity of the infection, the particular compound and its mode of administration.

IL-12 or inhibitor transforming growth factor-beta according to the invention are preferably formulated in unit dosage form to facilitate dosing administration and uniformity. In this document, the term “unit dosage form” refers to a physically distinct unit of compound suitable for the patient to be treated. However, it will be understood that the total daily dosage of the compounds and compositions according to the present invention will be decided by the attending physician. The specific effective dose level for a particular animal or human patient or subject will depend on a variety of factors including the disorder or disease being treated and the severity of the disorder or disease; The activity of the specific compound employed; The specific composition employed; Age, body weight, general health, sex and diet of the patient/subject; The period of administration, the route of administration and the rate of elimination of the specific compound employed; duration of treatment; The drugs used in combination or incidentally with the specific compound used and analogous factors well known in the medical arts. The term “patient” as used herein refers to an animal, preferably a mammal, and preferably a human.

According to the present invention, the pharmaceutical composition may comprise effective amount of Interleukin 12 (IL12) or derivative thereof. For example, the pharmaceutical composition may comprise doses Interleukin 12 (IL12) or derivative thereof adapted with regards to the nosocomial disease to be treated and/or to the subject to be treated. One skilled in the art taking into consideration his technical knowledge would adapt the amount in the pharmaceutical composition with regard to the nosocomial disease to be treated and/or to the subject to be treated. For example the pharmaceutical composition may comprise Interleukin 12 (IL12) at doses about 2 to 20 μg, preferably from 5 to 15 μg, preferably equal to 12.5 μg. For example, the pharmaceutical composition may comprise interleukin 12 (IL12) or derivative thereof in an amount allowing administration of IL-12 at doses of from about 0.1 μg/kg to 1 μg/Kg body weight of the subject.

According to the invention, interleukin 12 (IL12) or derivative thereof may be administered on a single administration or repeated administrations, for example one to three time per day.

According to the invention, interleukin 12 (IL12) or derivative thereof may be administered for example for a period from 1 to 21 days, for example from 1 to 7 days.

According to the present invention, the pharmaceutical composition may comprise any pharmaceutically acceptable and effective amount of inhibitor of transforming growth factor-beta. For example, the pharmaceutical composition may comprise doses of inhibitors of transforming growth factor-beta (TGF-β) adapted with regards to the inhibitor used. One skilled in the art taking into consideration his technical knowledge would adapt the amount in the pharmaceutical composition with regard to the used inhibitor. For example, when the inhibitors of transforming growth factor-beta (TGF-β) is small molecules, for example LY2157299, the pharmaceutical composition may comprise, for example at doses around 80 mg. For example, when the inhibitors of transforming growth factor-beta (TGF-β) is recombinant protein, for example Avotermin, the pharmaceutical composition may comprise, for example at doses from 20 ng to 200 ng, preferably from 50 ng to 200 ng, more preferably 100 ng to 200 ng. For example, when the inhibitors of transforming growth factor-beta (TGF-β) is humanized antibody, for example IMC-TR1, the pharmaceutical composition may comprise, for example at doses from 12.5 mg to 1600 mg.

According to the invention, inhibitors of transforming growth factor-beta (TGF-β) may be administered on a single time or repeated administration, for example one to three time per day.

According to the invention, inhibitors of transforming growth factor-beta (TGF-β) may be administered for example for a period from 1 to 21 days, for example from 1 to 7 days.

According to another aspect, the present invention relates to interleukin 12 (IL12) or derivative thereof, or pharmaceutical composition comprising IL12 or derivative thereof, for its use as a medicament, in particular in the treatment of secondary infection.

The Interleukin 12 (IL12) or derivative thereof is as defined above.

The pharmaceutical composition comprising IL12 or derivative thereof is as defined above.

The secondary infection is as defined above. For example secondary infection may be nosocomial diseases, including pneumonia, pleural infection, urinary infection, peritoneal infection, intra-abdominal abscess, meningitis, mediastinal infection, soft-tissue and/or skin infection, such as cellulitis.

According to another aspect, the present invention relates to an inhibitor of transforming growth factor-beta, or pharmaceutical composition comprising inhibitor of transforming growth factor-beta, for its use as a medicament, in particular in the treatment of secondary infection.

The inhibitor transforming growth factor-beta is as defined above.

The pharmaceutical composition comprising inhibitor transforming growth factor-beta is as defined above.

The secondary infection is as defined above. For example secondary infection may be nosocomial diseases, including pneumonia, pleural infection, urinary infection, peritoneal infection, intra-abdominal abscess, meningitis, mediastinal infection, soft-tissue and/or skin infection, such as cellulitis.

According to another aspect, the present invention relates to a method of treating or preventing secondary diseases comprising administering an effective amount of interleukin 12 (IL12) or derivative thereof or composition comprising interleukin 12 to a subject.

The Interleukin 12 (IL12) or derivative thereof is as defined above.

The composition comprising IL12 or derivative thereof is as defined above.

The secondary infection is as defined above. For example secondary infection may be nosocomial diseases, including pneumonia, pleural infection, urinary infection, peritoneal infection, intra-abdominal abscess, meningitis, mediastinal infection.

The administration of interleukin 12 (IL12) or derivative thereof or composition comprising interleukin 12 (IL12) or derivative thereof may be carried out by any way/routes known to the skilled person. For example it may be administered in any form and/or way/routes as mentioned above.

According to another aspect, the present invention relates to a method of treating or preventing secondary diseases comprising administering an effective amount of inhibitor of transforming growth factor-beta.

The inhibitor transforming growth factor-beta is as defined above.

The secondary infection is as defined above. For example secondary infection may be nosocomial diseases, including pneumonia, pleural infection, urinary infection, peritoneal infection, intra-abdominal abscess, meningitis, mediastinal infection

The administration of inhibitor transforming growth factor-beta or composition comprising inhibitor transforming growth factor-beta may be carried out by any way/routes known to the skilled person. For example it may be administered in any form and/or way/routes as mentioned above.

The medicament may be in any form that can be administered to a human or an animal. It may for example be a pharmaceutical composition as defined above.

The administration of the medicament may be carried out by any way known to one skilled in the art. It may, for example, be carried out directly, i.e. pure or substantially pure, or after mixing of the antibody or antigen-binding portion thereof with a pharmaceutically acceptable carrier and/or medium. According to the present invention, the medicament may be an injectable solution, a medicament for oral administration, for example selected from the group comprising a liquid formulation, a multiparticle system, an orodispersible dosage form. According to the present invention, the medicament may be a medicament for oral administration selected from the group comprising a liquid formulation, an oral effervescent dosage form, an oral powder, a multiparticle system, an orodispersible dosage form.

The interleukin 12 (IL12) or derivative thereof and/or inhibitor of transforming growth factor-beta as described above and pharmaceutically acceptable compositions of the present invention may also be used in combination therapies, i.e., compounds and pharmaceutically acceptable compositions may be administered simultaneously with, before or after one or more other therapeutic agents, or medical procedures. The particular combination of therapies (therapies or procedures) to be employed in an association scheme will take into account the compatibility of the desired therapeutic products and/or procedures and the desired therapeutic effect to be achieved. The therapies used may be directed to the same disease (for example, a compound according to the invention may be administered simultaneously with another agent used to treat the same disease), or may have different therapeutic effects (eg, undesirable).

For example, therapeutic agents known to treat secondary disease, for example nosocomial diseases, for example antibiotics, antifungal and/or antiviral compounds and/or antibacterial antibody and/or interferon therapy. It may be for example any antibiotic known to one skilled in the art. It may be for example antibiotic used for the treatment of pneumonia, pleural infection, urinary infection, peritoneal infection, intra-abdominal abscess, meningitis, mediastinal infection. It may be for example antibiotic selected from the group comprising Gentamicin, Kanamycin, Neomycin, Netilmicin, Tobramycin, Paromomycin, Streptomycin, Spectinomycin, Geldanamycin, Herbimycin, Rifaximin, Loracarbef, Ertapenem, Doripenem, Imipenem/Cilastatin, Meropenem, Cefadroxil, Cefazolin, Cefalotin or Cefalothin, Cefalexin, Cefaclor, Cefamandole, Cefoxitin, Cefprozil, Cefuroxime, Cefixime; Cefdinir; Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Ceftolozane, Avibactam, Teicoplanin, Vancomycin, Telavancin, Dalbavancin, Oritavancin, Clindamycin, Lincomycin, Daptomycin, Azithromycin, Clarithromycin, Dirithromycin, Erythromycin, Roxithromycin, Troleandomycin, Telithromycin, Spiramycin, Aztreonam, Furazolidone, Nitrofurantoin, Linezolid, Tedizolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Sulfonamidochrysoidine, Demeclocycline, Doxycycline, Minocycline, Oxytetracycline, Tetracycline, Clofazimine, Dapsone, Capreomycin, Cycloserine, Ethambutol(Bs), Ethionamide, Isoniazid, Pyrazinamide, Rifampicin, Rifabutin, Rifapentine, Streptomycin, Arsphenamine, Chloramphenicol(Bs), Fosfomycin, Fusidic acid, Metronidazole, Mupirocin, Platensimycin, Quinupristin/Dalfopristin, Thiamphenicol, Tigecycline(Bs), Tinidazole, Trimethoprim(Bs).

It may be for example antifungal compound selected from the group comprising Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Amphotericin B, Candicidin, Filipin, Hamycin, Natamycin, Nystatin, Rimocidin, Albaconazole, Efinaconazole, Epoxiconazole, Fluconazole, Isavuconazole, Itraconazole, Posaconazole, Propiconazole, Ravuconazole, Terconazole, Voriconazole, Abafungin, Anidulafungin, Caspofungin, Micafungin, Aurones, Benzoic acid, Ciclopirox, Flucytosine or 5-fluorocytosine, Griseofulvin, Haloprogin, Tolnaftate, Undecylenic acid.

It may be for example antiviral compound selected from the group comprising Abacavir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type Ill, Interferon type II, Interferon type I, Interferon, Lamivudine, Lopinavir, Loviride, Maraviroc Moroxydine, Methisazone, Nelfinavir, Nevirapine, Nexavir, Nitazoxanide, Nucleoside analogues, Novir, Oseltamivir, Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine.

The inventors have also demonstrated that the expression of transcription factor Blimp1 is increased in subject susceptible to secondary disease and/or nosocomial disease. In particular, the inventors have demonstrated that the expression of transcription factor Blimp1 is increased in subject with deficient or less reactive immune response to a pathogen.

Another object of the present invention is an ex vivo method for determining the immunity state of a subject comprising
a. Determining the level of expression of transcription factor Blimp1 (L_{dert}) in a biological sample of said subject,
b. Comparing the expression level of Blimp1 (L_{dert}) with a referenced level of expression of transcription factor Blimp1 (L_{ref}) by calculating a score S1=L_{dert}/L_{ref}
Wherein
   If S1>1, the subject is considered likely to have a deficiency in immunity response to any pathogen, or,
   If S1≤1, the subject is considered unlikely to have a deficiency in immunity response to any pathogen.

Another object of the present invention is an ex vivo method for determining the susceptibility to a secondary disease of a subject comprising
a. Determining the level of expression of transcription factor Blimp1 (L_{dert}) in a biological sample of said subject,
b. Comparing the expression level of Blimp1 (L_{dert}) with a referenced level of expression of transcription factor Blimp1 (L_{ref}) by calculating a score S1=L_{dert}/L_{ref}
Wherein
   If S1>1, the subject is considered likely to be susceptible to a secondary disease, or,
   If S1≤1, the subject is considered unlikely to be susceptible to a secondary disease.

In the present, “deficiency in immunity” means that the subject may have decreased immunogenic response and/or capacity of initiating adaptive and/or capacity of activating innate immunity with regards to a pathogen and/or a reduction of the activation or efficacy of the immune system.

In the present, “susceptibility to a secondary disease” means a subject having a reduction of the activation or efficacy of the immune system and/or having an increased susceptibility to opportunistic infections and decreased cancer immunosurveillance.

In other words, the method of the invention makes it possible to establish, before any secondary disease and/or nosocomial disease whether a subject may be more susceptible to such disease and whether the condition of a such can be improved by administration of a treatment, in particular a treatment improving and/or restoring the immunity response as the medicament of the invention i.e. IL-12 and/or inhibitor of TGF-β.

According to the invention, “biological sample” means a liquid or solid sample. According to the invention, the sample can be any biological fluid, for example it can be a sample of blood, of plasma, of serum, of cerebrospinal fluid, of respiratory fluid, of vaginal mucus, of nasal mucus, of saliva and/or of urine. Preferably the biological sample is a blood sample.

In the present by Blimp1 transcription factor is a protein that in humans is encoded by the PRDM1 gene.

According to the invention the expression level of transcription factor Blimp1 may be determined by any method or process known from one skilled in the art. It may be for example determined with flow cytomtery or any method disclosed in Marcel Geertz and Sebastian J. Maerkl, Experimental strategies for studying transcription factor—DNA binding specificities, Brief Funct Genomics. 2010 December; 9(5-6): 362-373 [39].

According to the invention the expression level of transcription factor Blimp1 may be determined from any immune cell of the biological sample. For example, the expression level of transcription factor Blimp1 may be determined from immune cell selected from the group comprising lymphocyte cells, phagocytes cells and granulocytes cells. It may be preferably determined from granulocytes selected from the group comprising macrophage, monocyte and dendritic cells. It may be preferably determined from dendritic cells.

According to the invention, the referenced level of expression of transcription factor Blimp1 (L_{ref}) may be the mean expression level of transcription factor Blimp1 (L_{ref}) in subject without any disease and/or which has not been infected with a pathogen at least since two weeks. For example the referenced level of expression of Blimp1 may be between 1000 to 100 000 gMFI in dendritic cells or less than 10% of Blimp1 positive dendritic cells or of B lymphocytes as measured by flow cytometry after intracellular staining.

The term “subject” as used herein refers to an animal, preferably a mammal, and preferably a human.

Other advantages may still be apparent to those skilled in the art by reading the examples below, illustrated by the accompanying figures, given by way of illustration.

### BRIEF DESCRIPTION OF THE FIGURES

FIG. 1 represents the recovery from primary pneumonia is followed by a susceptibility to secondary pneumonia and prolonged reduction in antigen presentation function. FIG. 1a. is a Schematic diagram of the experimental outline of primary (1 ary) and secondary (2ary) infection models with Escherichia coli (E. coli) or influenza A virus (IAV). FIG. 1b. is a Time course of the bacterial load after intra-tracheal instillation of E. coli in naive mice (primary pneumonia) (n=3 mice per group) in abscissa represent the day and ordinate log 10 of colony forming unit (c.f.u.) per milliliter (mL), FIG. 1c represent the Mouse weight loss during primary and secondary E. coli pneumonia realized 7 days after E. coli primary pneumonia (n=3 mice per group), ordinate represents the percentage of initial weight and abscissa the day after primary infection. FIG. 1d is Enumeration of colony-forming units per milliliter of bronchioalveolar lavage (c.f.u/mL) (ordinate) analyzed one day after secondary E. coli pneumonia (75 μL of DH5a intra-tracheal, OD600=0.6) induced at the indicated times after E. coli primary pneumonia (n=6 mice per group), abscissa represents the day between the primary and secondary infection. FIG. 1e is an Enumeration of c.f.u per milliliter of bronchoalveolar lavage (ordinate) analyzed one day after secondary E. coli pneumonia (75 μL of DH5α intra-tracheal) induced 7 days after influenza A virus (IAV) primary pneumonia (n=5 mice per group) (2ary PN realized after 7 d. after IAV). FIGS. 1f, 1g. At the indicated times after (f) E. coli or (g) IAV primary pneumonia, Cell Trace Violet-labeled OT-II cells (iv.) and OVA-coated E. coli (intra-tracheal) were injected concomitantly in WT mice. OT-II proliferation was assessed 60 hours later in the mediastinal lymph node (n=6-7 mice per group, except day 14 and day 21 n=3). On FIGS. 1f and g, ordinate represents the divided OT-II cells×10³ and abscissa the days between the first and secondary infection. *p<0.05, **p<0.01, # p<0.01 vs all infected groups, One-way ANOVA. Graphs represent mean+/−SD and are pooled data from 2-3 independent experiments. On

FIG. 2 demonstrates that Treg cells are induced by TGF-β and dampen CD4 T cell priming during infection-induced immunosuppression, and that blocking anti-TGF-β antibody restores lung response to secondary pneumonia. FIG. 2a. WT mice were treated with anti-TGF-α or isotype control monoclonal antibody after primary (1ary) E. coli pneumonia (44 μg i.p. at day+3 and day+6) then injected with E. coli at day+7 (2ary pneumonia (PN)). The number of colony forming units (c.f.u). per milliliter of bronchoalveolar lavage (BAL) (ordinate) was assessed 18 hours later (n 5 mice per group). FIG. 2b. WT mice were treated with anti-TGF-β or isotype control monoclonal antibody after primary (1ary) E. coli pneumonia (44 μg i.p. at day+3 and day+6), and subsequently injected (day+7) with OVA-coated E. coli (intra-tracheal) and Cell Trace Violet labeled OT-II (secondary, 2ary pneumonia). OT-II proliferation was assessed 60 hours later in the mediastinal lymph node (n=5-6 mice per group). FIG. 2c represents the frequency and number of lung FoxP3+CD4 T cells in WT mice that were uninfected (white dots), or infected with E. coli to elicit primary pneumonia (1ary PN, black dots), or following secondary E. coli pneumonia (2ary PN, grey dots) elicited 7 days following (I) E. coli or (II) IAV (each dot represents an independent biological replicate), abscissa represents the percentage of CD4 T cells and ordinate the number of FoxP3+CD4 T cells in Wild Type (WT) mice. FIG. 2d. Number and frequency of lung FoxP3+CD4 T cells in WT mice (ordinate) treated with anti-TGF-β or isotype control monoclonal antibody after primary pneumonia (1ary PN) (44 μg ip. at day+3 and day+6), and subsequently challenged (day+7) with secondary pneumonia (2ary PN) (n=5-6 mice per group). FIG. 2e. Number of c.f.u. per milliliter of bronchoalveolar lavage (BAL) during primary (1ary) or secondary (2ary) pneumonia in WT or DT-treated DEREG mice (DT 0.1 mg ip. day+4 and day+6 after 1ary pneumonia) (n=3-4 mice per group), ordinate represents the log 10 of c.f.u. per milliliter of bronchoalveolar lavage (BAL). FIG. 2f. 7 days E. coli primary pneumonia, WT or DT-treated DEREG mice (DT 0.1 mg ip. day+4 and day+6 after 1ary pneumonia) were subsequently injected with OVA-coated E. coli (intra-tracheal) and Cell Trace Violet labeled OT-II (secondary, 2ary pneumonia). OT-II proliferation was assessed 60 hours later in the mediastinal lymph node (n=5-6 mice per group), ordinate represents the number of divided OT-II cells×10³* p<0.05, ** p<0.01, # p<0.05 vs all others. Graphs represent mean+/−SD and are a compilation of 2-3 independent experiments.

FIG. 3 demonstrates that Macrophages and DC produce TGF-β in infection-cured mice, FIG. 3a. Relative expression of TGF-β mRNA in macrophages (ordinate) and DC purified by flow cytometry from lungs of WT mice infected or not with E. coli 7 days prior (infection-cured) (n=3 independent biological replicates per group from 4-5 pooled mice) in alveolar macrophage (Alveolar mac.), intern macrophage (Int mac), CD103 Dendritic cells (CD103 DC) and CD11b Dendritic cells (CD11b DC). FIG. 3b. Membrane expression of Latency Associated Peptide (LAP), inactive form TGF-β, by lung macrophages and DC from WT mice infected or not with E. coli or IAV 7 days prior and considered as infection-cured (n=4-6 mice per group), ordinate represents the percentage of cells expressing LAP in alveolar macrophage (LAP⁺ (% Alv. mac.)), intern macrophage ((LAP⁺ (% Int mac)), CD103 Dendritic cells ((LAP⁺ (% CD103 DC)) and CD11b Dendritic cells ((LAP⁺ (% CD11b DC)). FIG. 3c. Frequency and number of CD4+FoxP3+ Treg cells after 5 days of in vitro co-culture of naive OT-II cells (50×10³ cells) with soluble OVA and either (I) macrophages or (II) DC (10×10³ cells) isolated from lungs of naive mice or E. coli infection-cured mice (n=2 independent experiments with 5 pooled mice per biological replicate), ordinate represents the percentage of FoxP3+ cells among CD4 T lymphocytes (FoxP3+(% CD4 Tcells) or the number of FoxP3+CD4 (FoxP3+CD4 T cells (×10³)). FIG. 3d. Frequencies and number of lung FoxP3+ CD4 T cells in CD11c-DTR chimeric mice cured from E. coli infection, treated or not with DT (0.1 μg ip. day-1, day 0 then every 3 days) and injected or not with TGF-β treatment (1 μg i.p at day+6) (n=6-8 mice per group, except n=4 for TGF-β treatment), ordinate represents the number of FoxP3+CD4 (FoxP3+CD4 T cells (×10³) and abscissa the percentage of FoxP3+ cells among CD4 T lymphocytes (FoxP3+(% CD4 T cells). FIG. 3e. Frequency and number of lung FoxP3+CD4 T cells during secondary (2ary) E. coli pneumonia realized in CD11c-DTR mice treated or not with DT after cure from E. coli infection (0.1 μg i.p. day+6 and +7 after 1ary E. coli pneumonia) (each dot represents an independent biological replicate, n=5-6 mice per group). ordinate represents the number of FoxP3+CD4 (FoxP3+CD4 T cells (×10³) and abscissa the percentage of FoxP3+ cells among CD4 T lymphocytes (FoxP3+(% CD4 T cells)*p<0.05, **p<0.01. Graphs represent mean+/−SD and are a compilation of 2-3 independent experiments.

FIG. 4 demonstrates that transcriptional programming of newly formed macrophages and DC is altered locally after infection. FIG. 4a. Number of OT-II cells after 60 hours of in vitro co-culture of naive OT-II (50×10³ cells) with increasing doses of soluble OVA and either (a) macrophages or (b) DC (10×10³ cells) collected from lungs of naive mice or mice infected with E. coli 7 days prior (infection-cured) (n=2 independent experiments, data is pooled with 5 mice per group for macrophages and DC donors), ordinate represents the number of divided OT-Cells (×10³) and abscissa the concentration of soluble OVA (sOVA μg/mL). FIG. 4b. BrDU was injected i.p. (1 mg per day for 2 days) in uninfected WT mice, or in mice infected with E. coli 5-7 days earlier (infection-cured). Percentage of BrDU+ lung macrophages and DC was assessed by flow cytometry (n=3 mice per group), ordinate represents the Percentage of BrDU+ cells among lung macrophages or DC as stated. FIG. 4c. Cell Trace Violet-labeled OT-II cells (iv.) and anti-DEC205-OVA (intra-tracheal) were delivered to WT mice that were naive (uninfected) or infected 7 days prior with E. coli (infection-cured). OT-II proliferation was assessed 60 hours later in the mediastinal lymph node (n=5 mice per group), ordinate represents the number of divided OT-cells (×10³). FIG. 4d. Cell Trace Violet-labeled OT-II cells (i.v.) and E. coli (intra-tracheal) were administered to CD11c-OVA mice that were naive (1ary pneumonia) or E. coli infection-cured (2ary pneumonia). OT-II proliferation was assessed 60 hours later in the mediastinal lymph node (n=5 mice per group), ordinate represents the number of divided OT-cells (×10³). FIG. 4e,f. Frequencies of IL-12+CD103 DC, TNF-α+ alveolar macrophages and IL6⁺ CD11b DC (ordinate represents the corresponding percentage) during primary (1ary) E. coli pneumonia (75 μL of DH5α intra-tracheal, OD600=0.6) or secondary (2ary) E. coli pneumonia realized at the indicated time point after (e) E. coli or (f) Influenza A Virus (IAV) 1ary pneumonia (n>8 mice at day 7, n=2-3 at day 14, 21, 30 and 45). FIG. 4g. Enumeration of c.f.u from bronchoalveolar lavages 16 hours following intra-tracheal injection of E. coli (75 μL of DH5α intra-tracheal, OD600=0.6) in either naive mice (primary, 1ary pneumonia), or infection-cured (2ary pneumonia) with or without IL-12 treatment (100 ng i.p.) concurrent with induction of 2ary pneumonia (n=4-6 independent mice), ordinate represents the number of colony forming units (c.f.u). per milliliter of bronchoalveolar lavage (BAL). FIG. 4h. Expression of IRF-4 in lung DC of WT mice, and of ID2, Blimp1 and IRF8 in specific reporter mice (ID2GFP, Blimp1GFP and IRF-8YFP respectively) left uninfected or infected 7 days previously with E. coli (infection-cured). (n=6 for IRF-4, and =3-4 for reporter mice). FIG. 4i. Expression of IRF-4 in splenic DC of WT mice, and of ID2, Blimp1 and IRF8 in specific reporter mice left uninfected or infected 7 days previously with E. coli (infection-cured). (n=3-4 per group). FIGS. 4j-k. E. coli infection-cured mice were intravenously injected with (j) soluble OVA plus Cell Trace Violet labeled OT-II and OT-II proliferation in the spleen was assessed 60 hours later, ordinate represents the percentage of divided OT-cells, or (k) with CpG (20 nM i.v.) or LPS (1 μg i.v.) and frequency of splenic IL12⁺ CD8 DC was measured 2 hours later, ordinate represents the percentage of splenic IL12⁺ CD8 dendritic cells. *p<0.05, **p<0.01, ***p<0.001, # p<0.01 vs all other groups, One-way ANOVA. Graphs represent mean+/−SD and are pooled data from 2-3 independent experiments.

FIG. 5 demonstrates that TGF-β and Treg cells locally modulate the function of macrophages and DC following pathogen clearance of a primary pneumonia. FIG. 5 a. Frequency of IL12⁺ CD103 DC, IL12⁺ alveolar macrophages and IL6⁺ CD11 b DC was measured after induction of E. coli pneumonia in WT⁺Tlr9−/− mixed bone marrow chimeras (1:1 ratio) intratracheally injected (so-called secondary pneumonia, 2ary PN) or not (so-called primary pneumonia, 1 ary PN) with CpG 7 days prior (n=4 mice per group). FIG. 5 b-c. Percentage of IL12⁺ CD103 DC (ordinate) and IL6+CD11b DC (ordinate) during secondary (2ary) pneumonia (PN) elicited 7 days after primary (1ary) pneumonia in WT mice treated with anti-TGF- or isotype control monoclonal antibody (44 μg i.p.) (b) during (day+3 and day+6) or after (day+7 and day+10) resolution of 1 ary pneumonia (n=3-6 mice per group), ordinates represent the percentage of IL12+CD103 dendritic cells or of IL6+CD11 b dendritic cells or of TNFalpha. FIG. 5 d. Lethally irradiated WT recipient mice were reconstituted with a 3:1 ration of CD45.1⁺WT and CD45.2⁺ Tgfbr2^{fl/fl}ICd11^{ccre} (which produces TGF-βRII-deficient DC). Eight weeks after immune reconstitution, the percentage of CD45.2⁺ TGF-βRII-deficient macrophages (ordinate: % of TGF-β RII deficient cells (CD45.2⁺) and DC was assessed in the lungs of uninfected of E. coli infection-cured chimeras (n=4 mice per group). FIG. 5e. Lethally irradiated WT recipient mice were reconstituted with 3:1 H2^{-/-} (which produces MHC-II deficient cells unable to induce CD4 T cell proliferation) and CD45.2⁺ Tgfbr2fl/flCd11 ccre bone marrow. Eight weeks after immune reconstitution, Cell Trace Violet labeled OT-II (i.v.) and OVA-coated E. coli (intra-tracheal) were injected in naive (primary, 1 ary pneumonia) or E. coli infection-cured (secondary, 2ary pneumonia) chimeras. 60 hours later, the proliferation of OT-II was assessed in the mediastinal lymph nodes (n=5-6 mice per group) (ordinate represents the number of divided OT-II cells (×10³)). FIG. 5f. WT (CD45.1+): Tgfbr2^{flfl}Cd11^{ccre} (CD45.2⁺) chimeras E. coli infection-cured were re-challenged with E. coli (2ary pneumonia). Frequency of IL12+ alveolar macrophages, IL12⁺ CD103 DC and of IL6⁺ CD11 b DC was determined in WT and TGF-βRII-deficient cells (n=4 mice per group) (ordinates represent the percentage of IL12+ alveolar macrophages, IL12⁺ CD103 Dendritic Cells and of IL6⁺ CD11 b Dendritic Cells). FIG. 5 g. Frequency of IL12⁺ alveolar macrophages, IL12⁺ CD103 DC and of IL6⁺ CD11 b DC during primary (1ary) or secondary (2ary) pneumonia in wild-type (WT) or DT-treated DEREG mice (DT 0.1 mg i.p. day+4 and day+6 after 1ary pneumonia) (n>6 mice per group), ordinates represent the percentage of IL12+ alveolar macrophages, IL12⁺ CD103 Dendritic Cells and of IL6⁺ CD11 b Dendritic Cells).

*p<0.05, ***p<0.001, # p<0.05 vs all others. Graphs represent mean+/−SD and display data pooled from 2-3 independent experiments.

FIG. 6 demonstrates that Blimp1 expression in CD1c DC and Treg accumulation are correlated with the disease severity of humans presenting with systemic inflammatory response. FIG. 6a. Expression of Blimp1 in circulating CD1c DC and CD141 DC of uninfected donors and of patients presenting severe secondary infection (n=12 controls and n=5 patients with severe secondary infections). FIG. 6b. Expression of Blimp1 (ordinate in gMFI) in circulating CD1c DC collected in healthy controls and in brain-injured patients with trauma-induced systemic inflammation. Blood samples were collected 7 days after the trauma (n=15 controls and n=32 severe trauma patients). FIG. 6c-d. Correlation between Blimp1 expression in circulating CD1c DC and (c) trauma severity (Glasgow Coma Scale) or (d) duration of mechanical ventilation (days) in brain-injured patients with trauma-induced systemic inflammation. Glasgow Coma Scale rates the severity of the brain-injury from 15 (minor injury) down to 3 (major injury), ordinate represent the geometric mean of intensity (gMFI) of expression of Blimp1. FIG. 6e. Number (ordinate in μL) and frequency (ordinate in percentage total to Lymphocyte) of circulating CD4 Treg in brain-injured patients suffering from trauma-induced systemic inflammation. Blood samples were collected 1 and 7 days after the trauma (n=27 severe trauma patients), FIG. 6f. Correlation between the accumulation of Treg (Delta=number at day 7−number at day 1) and trauma severity in brain-injured patients (ordinate represents the Delta=number at day 7−number at day 1 in μL). Glasgow Coma Scale rates the severity of the brain-injury from 15 (minor injury) down to 3 (major injury). *p<0.05, **p<0.01, ***p<0.001. Graphs represent mean+/−SD.

FIG. 7 demonstrates the Effect of blocking anti-TGF-β antibody on the magnitude of Escherichia coli primary pneumonia. Mice treated with anti-TGF-β or isotype control monoclonal antibody after primary E. coli pneumonia (44 μg i.p. at day+3 and day+6). FIG. 7 a, Time course of the mice weight (ordinate in percentage) over the 7 days (abscissa) following the infection. (n=6 mice per group). Experiment performed once. FIG. 7b, the number of colony forming units (c.f.u). per milliliter of bronchoalveolar lavage (BAL) (ordinate) was assessed 18 hours after E. coli pneumonia, and 24 hours after each injection of antibody (day+4 and day+7).(n=3-4 mice per group). Experiment performed once. FIG. 7 c-d, CD86 expression (ordinate in gMFI) by (c) dendritic cells and (d) macrophages was assessed 18 hours after E. coli pneumonia, and 24 hours after each injection of antibody (day+4 and day+7).(n=3 mice per group). Experiment performed once. **p<0.01

FIG. 8 represents Treg and TGFβ left over in infection-cured mice. FIG. 8 a, b. Frequencies and number of lung FoxP3 CD4 T cells (ordinate: number of cells×10³) in wild type mice uninfected or infected with (a) Escherichia coli (E. coli) (black circles) or (b) Influenza A Virus (black circles) (IAV) 7 days prior and considered as cured from infection. FIG. 8 c, DEREG mice were injected with E. coli (75 μL intra-tracheal, OD₆₀₀=0.6), treated or not with diphtheria toxin (day+4 and day+7) then challenged with secondary (2ary) E. coli. The number of FoxP3+CD4 T (ordinate: number of cells×10³) cells was assessed 18 hours later in the lungs. (n=6 mice per group). Representative of 2 independent experiments. FIG. 8d, Time course of the mice weight (ordinate: % of initial weight) over the 7 days (abscissa) following the infection in WT or in DEREG mice treated with DT (DT 0.1 mg ip. day+4 and day+7). (n=3 mice per group). Experiment performed twice. FIG. 8e, Number of c.f.u. per milliliter of bronchoalveolar lavage (BAL) (ordinate) during primary (1ary) in WT or DT-treated DEREG mice (DT 0.1 mg ip. day+4 and day+7) (n=3 mice per group). Experiment performed once. FIG. 8 f, g. CD86 expression (ordinate in gMFI) by (f) dendritic cells and (g) macrophages was assessed 18 hours after E. coli pneumonia, and 24 hours after each injection of Diphteria toxin (day+4 and day+7). (n=3 mice per group). Experiment performed once. FIG. 8h, Relative expression of mRNA of TGFβ (ordinate) in CD45 cells (abscissa) sorted from the lungs of naive mice (empty baton) or mice infected 7 days prior (infection-cured) with E. coli (filed baton). (n=8 biological replicates from 2 independent experiments (2 pooled mice per biological replicate). FIG. 8i, Frequencies of neuropilin⁺ FoxP3⁺ CD4 T cells (ordinate in percentage) in uninfected mice (empty baton), or mice infected with E. coli infection-cured. (n=5-8 mice per group) (filed baton). FIG. 8j, Relative expression of mRNA of aldh1a2, Integrin b8 (Itgb8) and PLAT (ordinate) in macrophages and DC sorted from the lungs of naive mice (empty baton) or mice infected 7 days prior (infection-cured) with E. coli. (filed baton) (n=3 independent experiments with 5 pooled mice per biological replicate).

FIG. 9 represents CD11c+ cells response to primary pneumonia. FIG. 9a, Phenotypic analysis of lungs collected 7 days after an E. coli pneumonia in wild-type and CD11c-diptheria toxin receptor chimeric mice treated with diphtheria toxin (0.1 μg ip., day-1, day 0, day+3 and day+6). FIG. 9b, c. Number of alveolar macrophages, interstitial macrophages and DCs (ordinate: number of cells×10³), and (c) of NK cells and CD4 T cells (ordinate: number of cells×10³) 7 days after E. coli pneumonia in the lungs of CD11c-DTR mice treated or not with diphtheria toxin (0.1 μg ip., day-1, day0, day+3 and day+6). (n=6 mice per group). Data are representative of 2 independent experiments. *p<0.005; **p<0.01.

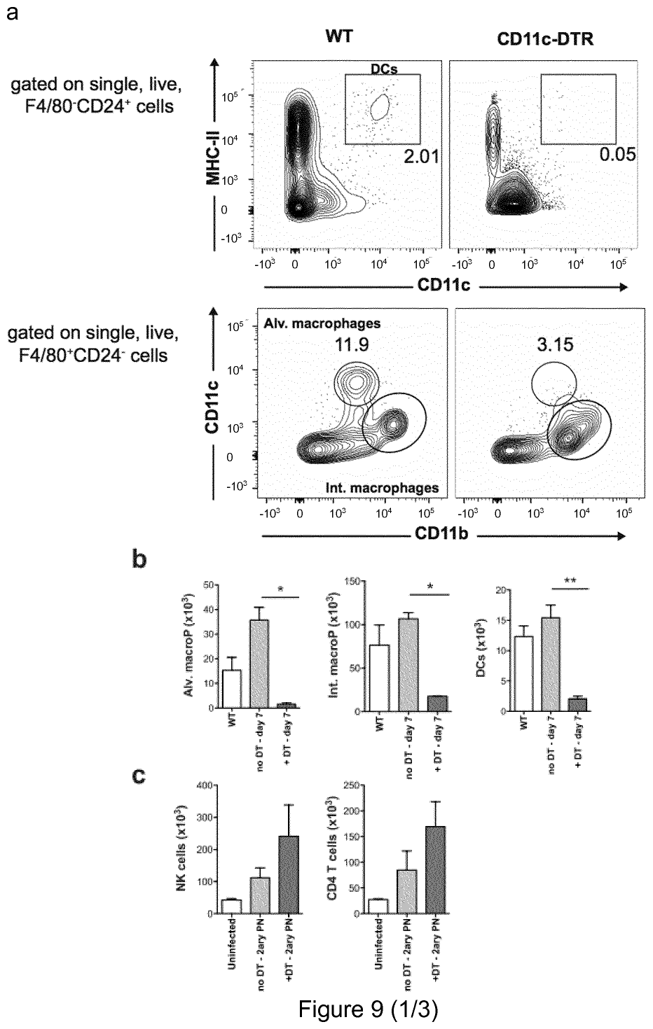

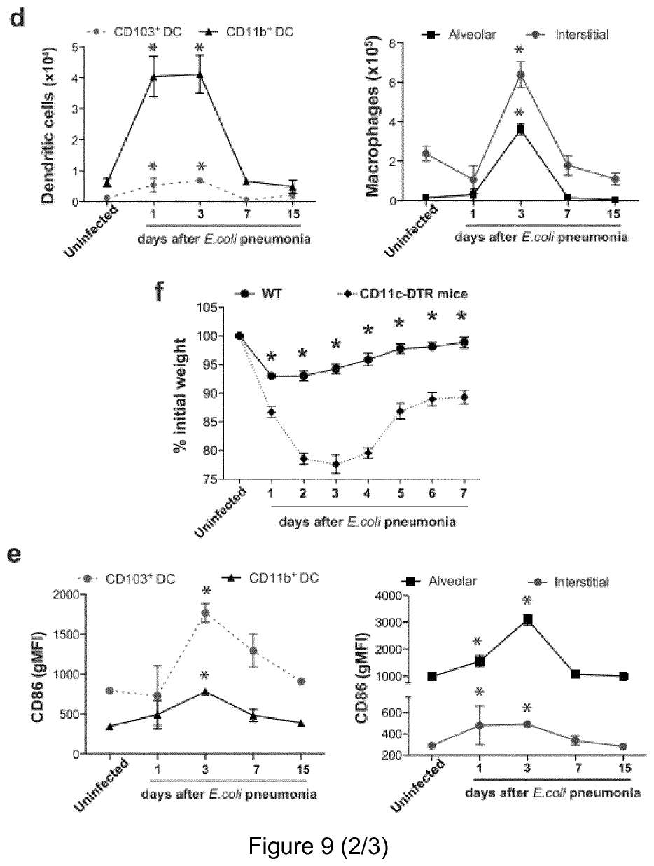

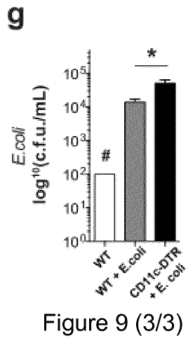

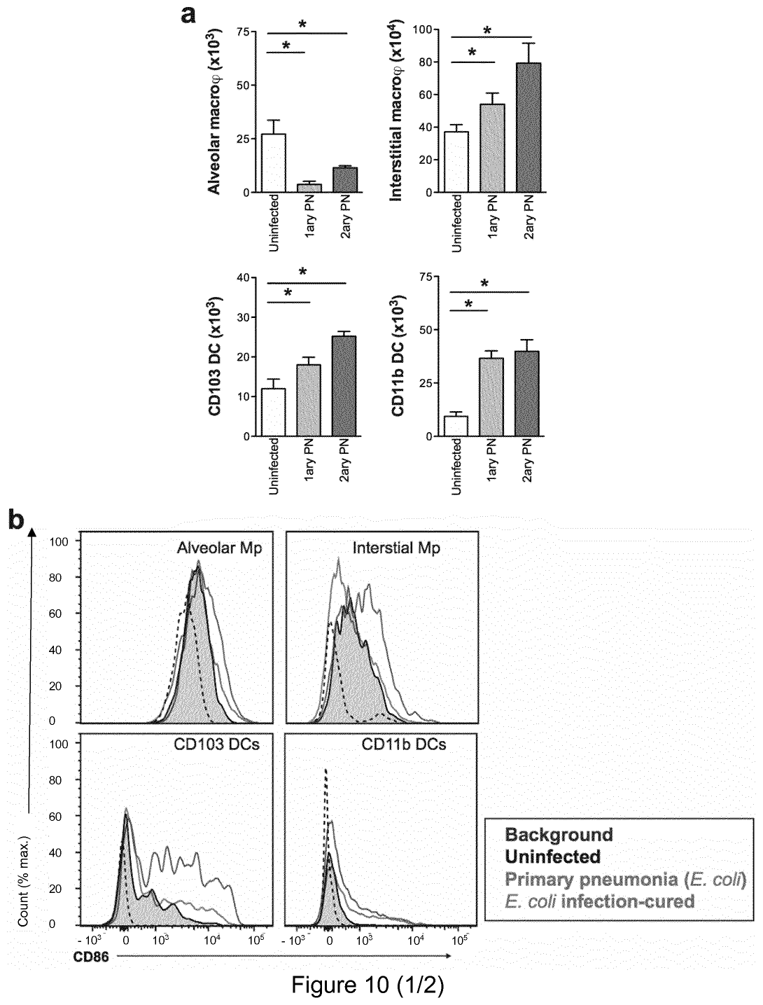

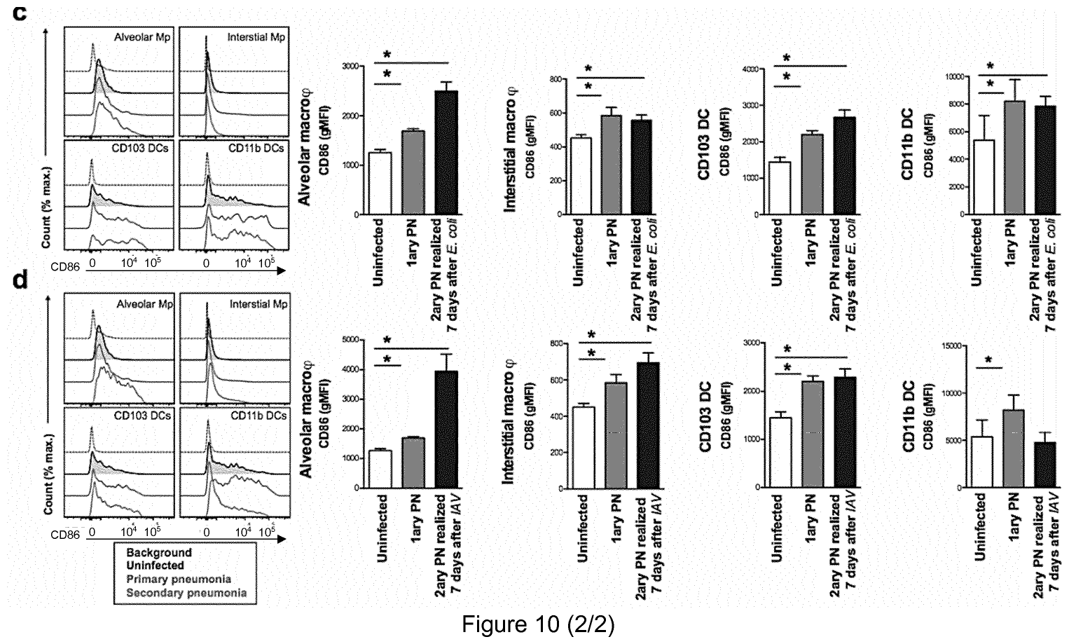

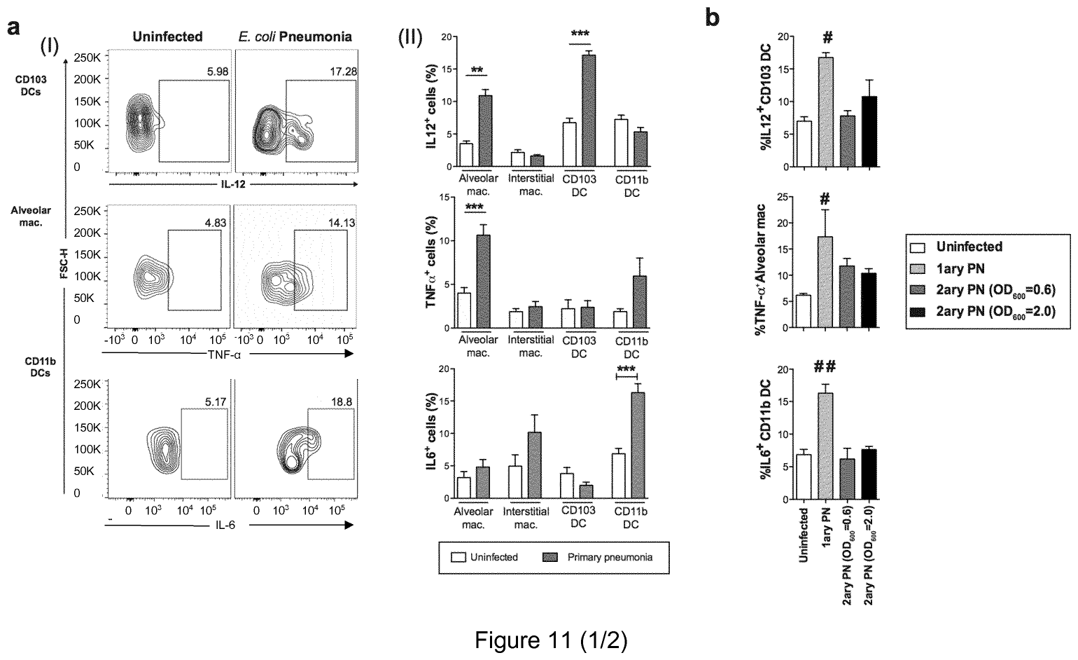

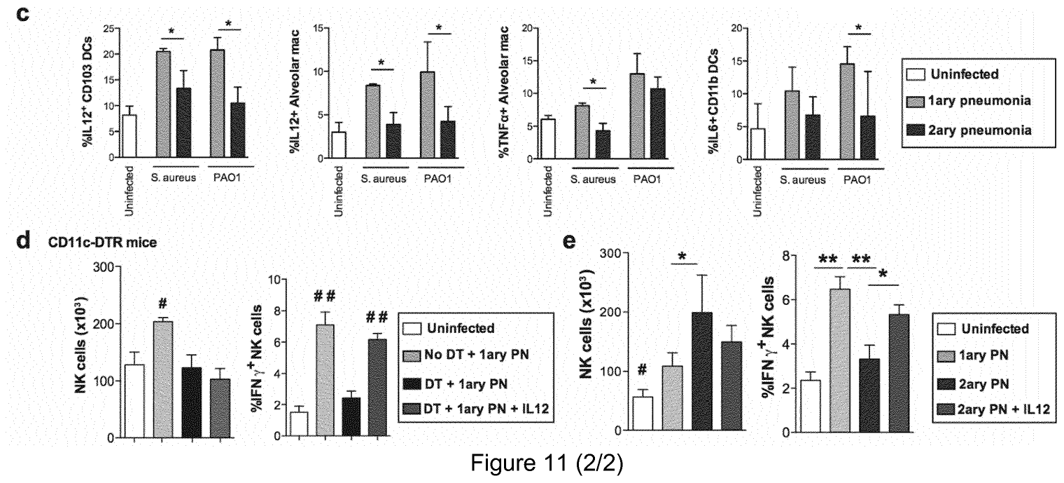

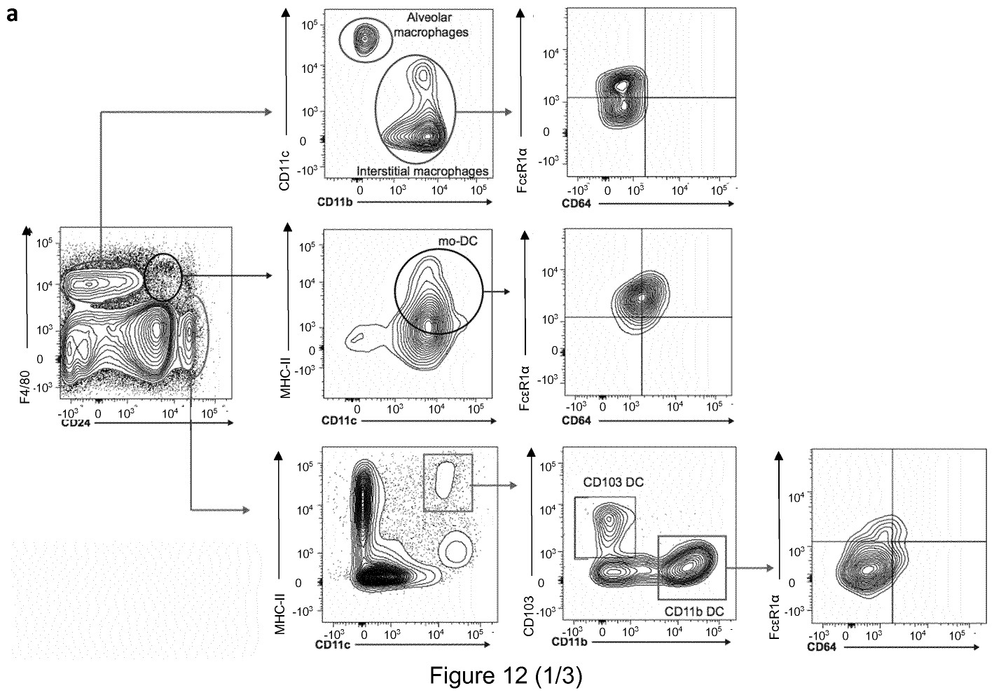

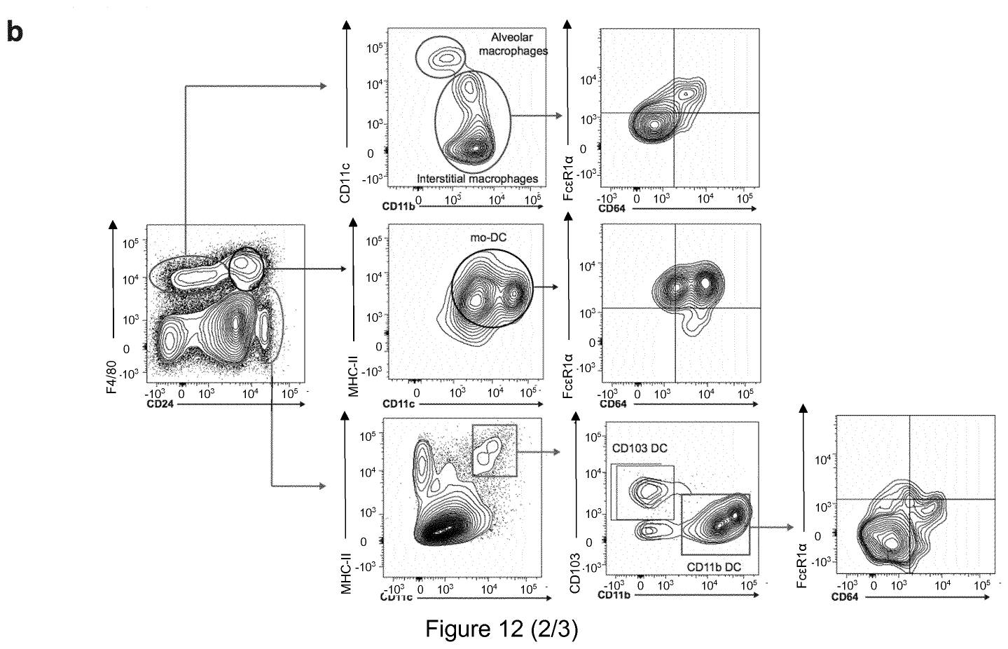

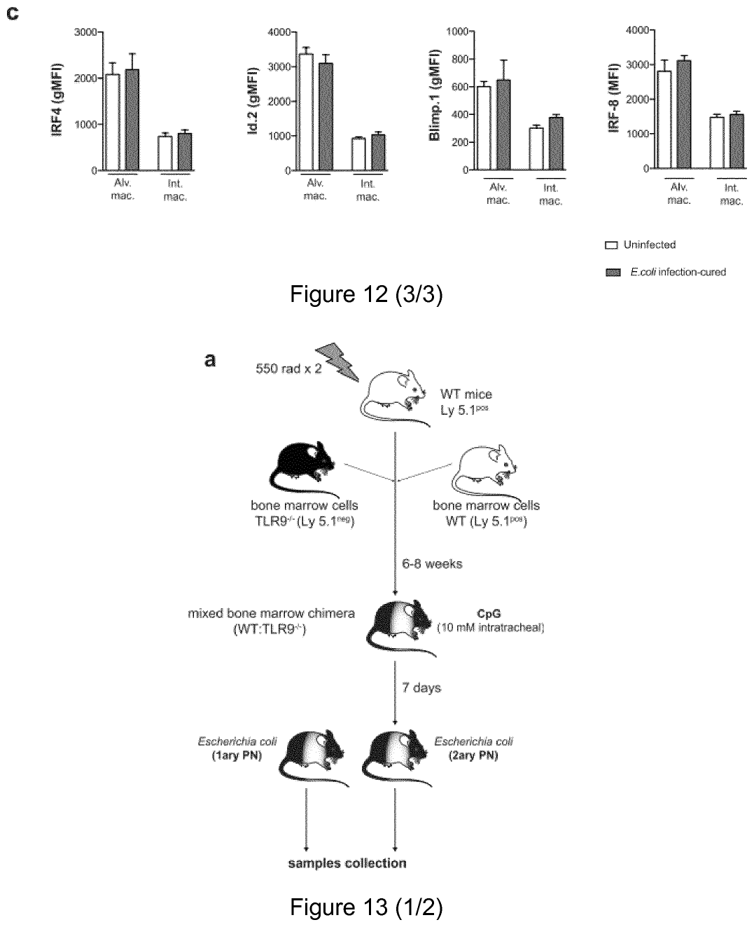

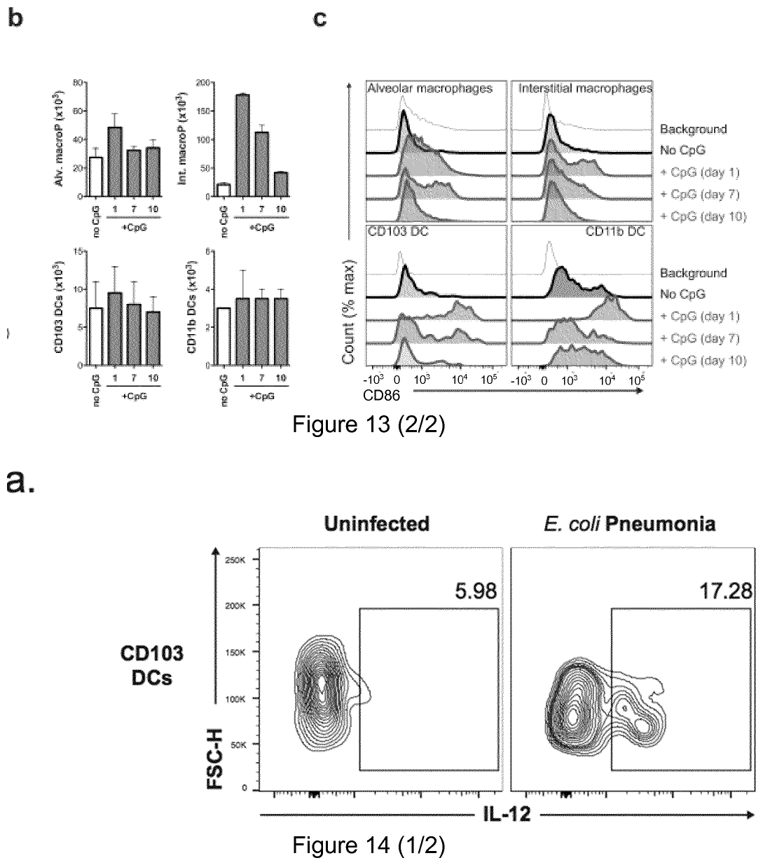

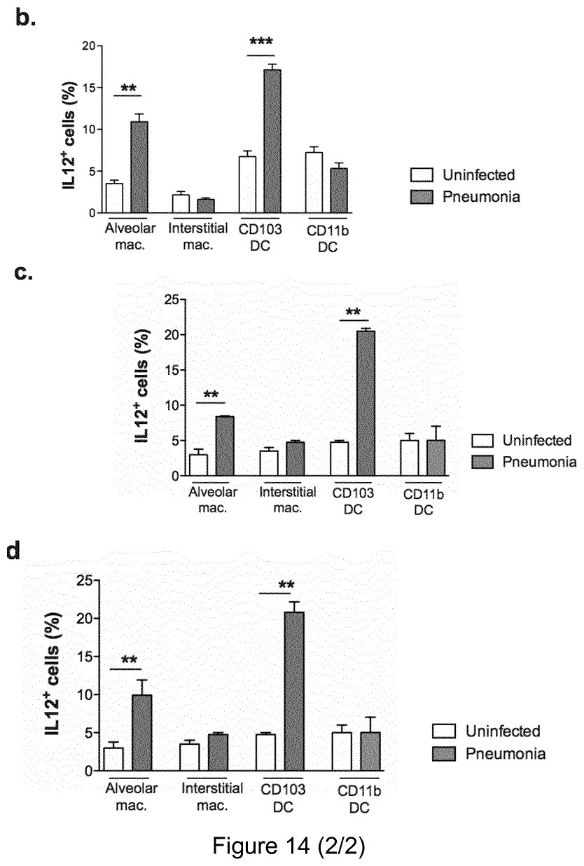

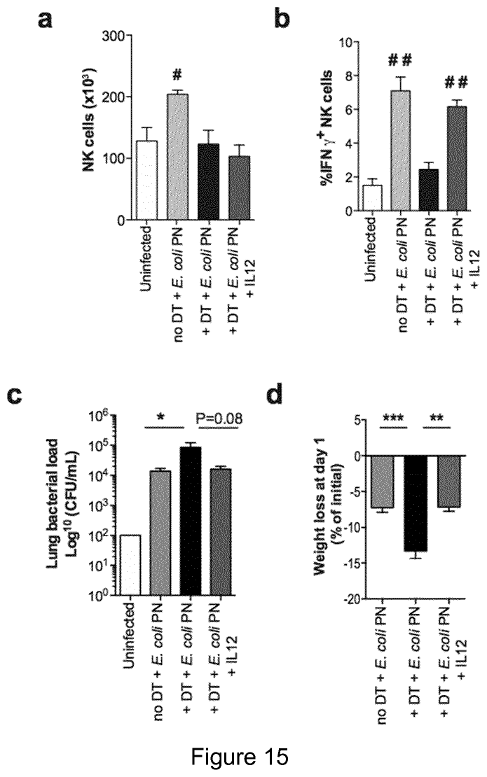

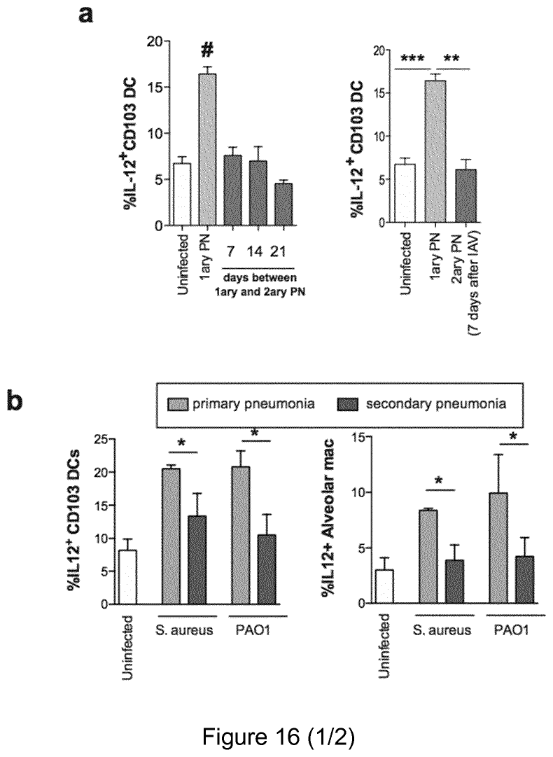

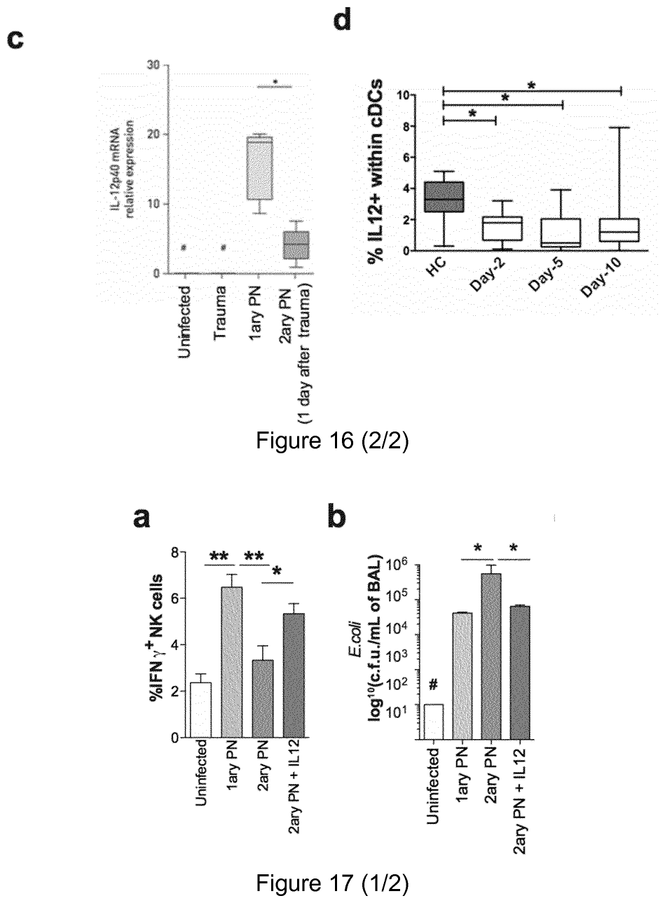

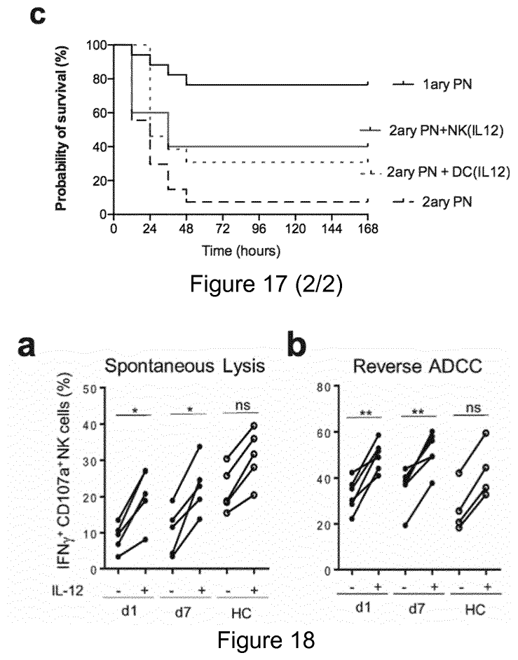

Graphs illustrate FIG. 9 (d) absolute numbers (ordinate: number of cells×10³) and FIG. 9 (e) CD86 expression (ordinate in gMFI) of alveolar macrophages, interstitial macrophages, CD103 DC and CD11 b DC at indicated time points after Escherichia coli (E. coli) induced pneumonia. Graphs represent mean+/−SD and display data pooled from 2 independent experiments. (n=4-6 mice per time point). *p<0.05 vs uninfected. FIG. 9 f, g. Weight loss (ordinate: percentage of initial weight) and (g) enumeration of c.f.u from bronchoalveolar lavages (at day 1) following E. coli intra-tracheal administration in WT mice and in DT-treated CD11c-DTR chimeric mice (ordinate: log 10 of c.f.u/mL of E. coli.) Graphs represent mean+/−SD and display data pooled from 2-3 independent experiments. (n=6 mice per group) # p<0.05 vs uninfected. *p<0.05.

FIG. 10 represents the Number and CD86 expression of lung macrophages and dendritic cells during primary and secondary pneumonia. FIG. 10 a, Number of alveolar macrophages, interstitial macrophages, CD103 DCs and CD11 b CDCs (ordinate: number of cells×10³) in the lungs of uninfected mice, or 16 hours after the onset of an E. coli primary pneumonia (1ary PN) or 16 hours of the onset of an E. coli pneumonia realized 7 days after a 1ary E. coli PN (2ary PN). (n=6 mice per group). Data are representative of 3 independent experiments. *p<0.05 FIG. 10 b, CD86 expression on alveolar macrophages, interstitial macrophages, CD103 DC and CD11 b DC in the lungs of uninfected mice, during primary pneumonia (day 3) or 7 days after E. coli pneumonia (infection-cured). (n=5-6 mice per group). Data are representative of 2 independent experiments. FIG. 10 c-d, Uninfected mice, or mice infected with (c) E. coli or (d) influenza A virus (IAV) 7 days previously, were challenged with E. coli (secondary pneumonia). Macrophages and dendritic cells were analyzed for CD86 expression (ordinate in gMFI) 16 hours after E. coli administration. Dotted line corresponds to background staining. (n=5-6 mice per group). Data are representative of 2 independent experiments. *p<0.05

FIG. 11 represents Cytokine production by CD11c+ cells during primary and secondary E. coli pneumonia. Mice were challenged with E. coli (75 μL intra-tracheal, OD₆₀₀=0.6). Eighteen hours later, frequencies of cytokine cells were determined by intracellular staining and flow cytometry analysis. FIG. 11a, (i) Representative flow cytometry plots for the percentage of cytokine expressing macrophages or dendritic cells in uninfected or infected mice. (ii) Frequencies of IL12⁺, TNFa⁺, and IL6⁺ lung macrophages and DC in mice injected (1ary pneumonia) or not (uninfected) with E. coli (intra-tracheal) 16 hours prior, (ordinate represents the percentage of IL6+, TNFα+ or IL12+ cells). Data are representative of more than 5 independent experiments. FIG. 11b, Frequencies of IL12+ CD103 dendritic cells (DC), TNF-a+ alveolar macrophages (mac) and IL6+ CD11 b DC during secondary (2ary) pneumonia with a low (OD600=0.6) or high (OD₆₀₀=2.0) dose of E. coli injected 7 days after primary (1ary) pneumonia (OD600=0.6) n=3 mice per group, (ordinate represents the percentage of IL6+ CD11 b DC, TNFα+ alveolar macrophages or IL12+ CD103 dendritic cells). Experiment performed once. FIG. 11c, Frequencies of IL-12⁺ CD103 D or alveolar macrophages, TNF-a⁺ alveolar macrophages and IL6 CD11 b DC during S. aureus pneumonia (ATCC 29280) or P. aeruginosa (PAO1) induced in naive mice (primary pneumonia) or in E. coli infection-cured mice (secondary pneumonia). (n=2-3 mice per group), (ordinate represents the percentage of IL12+ CD103 dendritic cells, IL12+ alveolar macrophages, TNFα+ alveolar macrophages or IL6+CD11b dendritic cells). Experiment performed once. FIG. 11 d, Number of NK cells (ordinate: number of cells×10³) and frequencies of IFN-γ⁺ NK cells (ordinate: percentage of IFN-γ⁺ NK cells) in uninfected or infected (E. coli 1ary PN) CD11c-DTR chimeric mice treated or not with diphtheria toxin (DT) for depletion of CD11c+ cells and interleukin (IL)-12.(n=4-6 mice per group). Data pooled from 2 independent experiments. FIG. 11e, Number of NK cells (ordinate: number of cells×10³) and frequencies of IFN-γ⁺ NK cells (ordinate: percentage of IFN-γ⁺ NK cells) in wild type mice uninfected, undergoing primary (1ary) E. coli pneumonia, secondary (2ary) pneumonia induced 7 days after 1ary pneumonia treated or not with IL-12. (n=4-6 mice per group). Data are representative of 2 independents experiments. Graphs represent mean+/−SD.*p<0.05, **p<0.01, **p<0.001. # p<0.05; ## p<0.01 vs all others.

FIG. 12 demonstrates phenotypic analysis and transcriptional program of lung macrophages and dendritic cells in uninfected or E. coli infection-cured mice. FIG. 12 a-b, Representative gating for the analysis of alveolar macrophages, interstitial macrophages, monocyte-derived dendritic cells (mo-DC), CD103 and CD11b dendritic cells in (a) uninfected mice or in (b) infection-cured mice (infected 7 days prior with E. coli). FIG. 12 c, Expression of IRF-4 (ordinate in gMFI) in lung macrophages of wild type mice, and of ID2 (ordinate in gMFI), Blimp1 (ordinate in gMFI) and IRF8 (ordinate in MFI) in specific reporter mice left uninfected or infected 7 days previously with E. coli (infection-cured).

FIG. 13 demonstrates CpG-induced lung inflammatory response and generation of wild-type (WT):TLR9^{-/-} mixed bone marrow chimeras. FIG. 13 a, Experimental diagram for the generation of WT:TLR9^{-/-} mixed bone-marrow chimeras, injected with CpG (10 nM intra-tracheal) and E. coli 7 days later (75 μL intra-tracheal, OD₆₀₀=0.6). FIG. 13 b, c, Graphs illustrate the absolute number (b) (ordinate: number of cells×10³) and CD86 expression (c) of alveolar macrophages, interstitial macrophages, CD103 DC and CD11b DC at indicated time points after intra-tracheal instillation of CpG in wild type mice. n=2-3 mice per time point. Data representative of 2 independents experiments.

FIG. 14 demonstrates that alveolar Macrophages and CD103 dendritic cells are the main source of IL-12 during bacterial pneumonia Wild type mice were challenged with (a,b) E. coli (75 μL intra-tracheal, OD₆₀₀=0.6),(c) S. aureus (75 μL intra-tracheal, OD₆₀₀=0.6) or (d) P. aeruginosa (75 μL intra-tracheal, 1/10 of OD₆₀₀=2.0). Eighteen hours later, frequencies of cytokine' cells were determined by intracellular staining and flow cytometry analysis. FIG. 14 (a) Representative flow cytometry plots for the percentage of IL12 expressing CD103+ dendritic cells in uninfected or E. coli-infected mice. FIG. 14 (b-d) Frequencies of IL12+ cells (ordinate: percentage of IL12+ cells) in mice injected (1ary pneumonia) or not (uninfected) with (b) E. coli, (c). S. aureus or (d) P. aeruginosa. Data are representative of more than 5 independent experiments. Graphs represent mean+/−SD.**p<0.01, **p<0.001.

FIG. 15 represents Production of IL-12 by dendritic cells and macrophages is critical for the innate immune response and clinical recovery to bacterial pneumonia

Macrophages and dendritic cells were depleted in vivo by treating CD11c-DTR mice with diphteria toxin. (a) Number of NK cells (ordinate: number of cells×10³) and (b), frequencies of IFN-γ+ Natural Killer cells (ordinate: percentage of IFN-γ+ Natural Killer cells) in uninfected or infected (E. coli intra-tracheal) mice depleted in macrophages and dendritic cells and treated or not with interleukin (IL)-12 (100 ng. ip.) n=4-6 mice per group). (c) Enumeration of colony forming units from bronchoalveolar lavages (ordinate: log 10 of CFU/mL) and (d) weight loss (ordinate: percentage of initial weight) 18 hours after E. coli intra-tracheal administration mice depleted in macrophages and dendritic cells and treated with

interleukin (IL)-12 (n=4-5 mice per group).

FIG. 16 represents the production of IL-12 by macrophages and dendritic cells is drastically decreased during bacterial pneumonia in mice and in humans cured from a primary infection or after non-septic inflammatory response (such as trauma, brain-injury). FIG. 16 (a) Frequencies of IL-12⁺ CD103 dendritic cells (ordinate: percentage of IL-12⁺ CD103 dendritic cells) during primary (1ary) E. coli pneumonia or secondary (2ary) E. coli pneumonia realized at the indicated time point after (i) E. coli or (ii) Influenza A Virus (IAV) 1ary pneumonia (n>8 mice at day 7, n=3 at day 14 and n=2 at day 21). FIG. 16 (b) Frequencies of IL-12⁺ CD103 dendritic cells (left panel, ordinate: percentage of IL-12⁺ CD103 dendritic cells) and of IL-12⁺ alveolar macrophages (right pane, ordinate: percentage of IL-12⁺ alveolar macrophages) during S. aureus pneumonia or P. aeruginosa induced in naive mice (primary pneumonia) or in E. coli infection-cured mice (secondary pneumonia). FIG. 16 c) mRNA levels of IL12 (ordinate: relative expression as compared to Sham) in conventional dendritic cells during S. aureus pneumonia in naive mice (primary pneumonia, 1ary PN) or in trauma-hemorrhage mice (secondary pneumonia, 2ary PN). FIG. 16 d) Frequencies of IL-12⁺ conventional DC (ordinate: percentage of IL-12⁺ dendritic cells) upon in vitro stimulation with LPS of peripheral blood mononuclear cells harvested in healthy controls (HC) and in critically ill patients at the indicated time after acute brain-injury.

FIG. 17 represents that IL-12 treatment restores innate immune response and enhances clinical recovery during bacterial pneumonia in mice cured from infection or from trauma-hemorrhage. FIG. 17 (a) frequencies of IFN-γ+ NK cells (ordinate: percentage of IFN-γ+ NK cells) in mice uninfected, undergoing primary (1ary) E. coli pneumonia, secondary (2ary) pneumonia induced 7 days after 1ary pneumonia or IL-12 (100 ng ip.) for the treatment of 2ary pneumonia. FIG. 17 (b) Enumeration of colony forming unit from bronchoalveolar lavages (ordinate: log 10 of c.f.u./mL of bronchoalveolar lavages (BAL)) 18 hours after E. coli intra-tracheal administration infection-cured mice challenged with a secondary pneumonia and treated or not with IL-12 (100 ng i.p. day 0) (n=4-5 mice per group). FIG. 17 (c) Survival curves to S. aureus pneumonia induced in naive mice (1ary PN), in trauma-hemorrhage mice (2ary PN), in trauma-hemorrhage mice injected with NK cells treated ex vivo with IL-12 (2ary PN+NK(IL12)) or injected with DCs producing IL12 (ordinate: percentage of probability of survival, abscissa: time in hours).

FIG. 18 represents NK cells of critically ill patients susceptible to bacterial pneumonia remain responsive to IL-12 treatment Frequencies of IFNγ+ CD107a+ NK cells (ordinate: percentage of IFNγ+ CD107a+ NK cells) in a 5-hours functional assay following the PBMC treated or not overnight with IL12. (i) spontaneous lysis with and without O/N treatment with IL-12 of PBMC of traumatic-brain injured patients on days 1 (d1) (n=5) and day 7(d7) (n=5) and healthy controls (HC) (n=5) and (ii) reverse ADCC with and without O/N treatment with IL-12 of PBMC of TBI on days 1 (n=6) and 7 (n=6) and HC (n=4)

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

The present invention and its applications can be understood further by the examples that illustrate some of the embodiments by which the inventive medical use may be reduced to practice. It will be appreciated, however, that these examples do not limit the invention. Variations of the invention, now known or further developed, are considered to fall within the scope of the present invention as described herein and as hereinafter claimed.

### EXAMPLES

### Example 1: Effect of IL-12 and TGF-β Inhibitors on Nosocomial Disease and Biological Mechanism Involved

### Material and Methods

Mice used were C57BL/6J (B6), B6.SJL-Ptprc^{a}Pep3^{b}/BoyJ (CD45.1), B6.FVB-Tg(ltgax-DTR/EGFP)57Lan/J (CD11c-DTR mice, Diphteria Toxin Receptor is expressed under the control of ltgax promoter) (Jung et al., 2002 [29]), C57BL/6J-Tlr9M7Btlr/Mmjax (Tlr9^{-/-} mice) (Hemmi et al., 2000 [25]), B6.Cg-Tg(TcraTcrb)425Cbn/J (OT-II mice) (Barnden et al., 1998 [7]), C57/B6.129S2-H2^{dIAb}1-Ea/J (H2 mice) (knock out for MHC-II gene)(Köntgen et al., 1993 [34]), CD11c-OVA (membrane OVA is expressed under the control of Itgax promoter) (Wilson et al., 2006 [66]), C57BL/6-Tg(Foxp3-DTR/EGFP)23.2Spar/Mmjax (Diphteria Toxin Receptor and GFP are expressed under the control of FoxP3 promoter, DEREG)(Lahl et al., 2007 [35]),ID2^{GFP} reporter (GFP is expressed under the control of ID2 promoter) (Jackson et al., 2011 [28]), Blimp1^{GFP} reporter (GFP is expressed under the control of Blimp1 promoter) (Kallies et al., 2004 [30]), IRF8^{YFP} (YFP is expressed under the control of IRF8 promoter) (Chopin et al., 2013 [19]), and Tgfb2r^{fl/fl} (Floxed regions around Tgfb2r gene) (Ramalingam et al., 2012 [44]) crossed to CD11c^{cre} (in which Cre recombinase is expressed under the control of the CD11c promoter) (Caton et al., 2007 [13]).

For technical reasons, mice were used for experiments without taking gender into account. Male and female mice were maintained in specific pathogen-free conditions, group housed, at the Bio21 Institute Animal Facility (Parkville, Australia) following institutional guidelines and were used for experiments between six and fourteen weeks of age. Experimental procedures were approved by the Animal Ethics Committee of the University of Melbourne (protocol #1413066).

Bioresources: IBIS-sepsis (severe septic patients) and IBIS (brain-injured patients), Nantes, France. Patients were enrolled from January 2014 to May 2016 in two French Surgical Intensive Care Units of one university hospital (Nantes, France). The collection of human samples has been declared to the French Ministry of Health (DC-2011-1399), and it has been approved by an institutional review board. Written informed consent from a next-of-kin was required for enrolment. Retrospective consent was obtained from patients, when possible.

For the IBIS-septic study, inclusion criteria were proven bacterial infection, together with a systemic inflammatory response (two signs or more among increased heart rate, abnormal body temperature, increased respiratory rate and abnormal white-cell count) and acute organ dysfunction and/or shock. For the IBIS study, inclusion criteria were brain-injury (Glasgow Coma Scale (GCS) below or equal to 12 and abnormal brain-CT scan) and systemic inflammatory response syndrome. Exclusion criteria were cancer in the previous five years, immunosuppressive drugs and pregnancy. All patients were clinically followed up for 28 days. Control samples were collected from matched healthy blood donors (age±10 years, sex, race), recruited at the Blood Transfusion Center (Etablissement Français du Sang, Nantes, France).

EDTA-anticoagulated blood samples were withdrawn seven days after primary infection in septic patients (IBIS sepsis), or at day 1 and day 7 ICU admission in trauma patients. Peripheral blood mononuclear cells (PBMCs) were isolated by centrifugation, frozen in liquid nitrogen in a 10% DMSO solution and stored until analysis.

### CD11c Cells (Macrophages and Dendritic Cells) and Treg Cell Depletion

Diphteria toxin (0.1 μg i.p, two injections 24 hours apart, then every 3 days) was administrated to CD11c-DTR or FoxP3^{GFP}-DTR (DEREG) mice to induce depletion of CD11c⁺ cells or Treg cells respectively. For CD11c-DTR mice, the first DT injection was performed either one day before the primary pneumonia (for outcomes assessed during primary infection or 7 days after), or 1 day before the secondary pneumonia as stated. DEREG mice were treated from day 4 after the primary pneumonia. Efficiency of depletion (number of cells) was controlled during experiments and routinely exceeded 90%.

### Induction of Primary and Secondary Pneumonia

Primary pneumonia, Escherichia coli (DH5a), grown for 18 hours in luria broth medium at 37° C., was washed twice (1.000× g, 10 min, 37° C.), diluted in sterile isotonic saline and calibrated by nephelometry. As stated, E. coli (75 μL, OD₆₀₀=0.6-0.7, or OD₆₀₀=2.0) or Influenza-virus (400 plaque-forming units of influenza, virus strain WSN x31) were injected intra-tracheally or intra-nasally respectively in anesthetized mice to induce a non-lethal acute pneumonia (Broquet et al., 2014; Wakim et al., 2013). For the secondary pneumonia, E. coli (75 μL, OD₆₀₀=0.6-0.7) or OVA-coated E. coli (see preparation below, 70 μL, OD₆₀₀=0.6-0.7) or Staphylococcus aureus (ATCC 29213, 70 μL, OD₆₀₀=0.6-0.7) or Pseudomonas aeruginosa (PAO1, 70 μL, 1/10 dilution of a solution OD₆₀₀=0.2-0.3) were injected intra-tracheally 7 to 21 days after the primary pneumonia.

### Induction of Aseptic Lung Inflammatory Response

CpG 1668 (10 nM) was administrated intra-tracheally under anaesthesia. Mice were kept in a semi-recumbent position for 60 seconds after injection.

### Generation of Mixed Bone Marrow Chimeras

Recipient mice were γ-irradiated twice with 550 Gray and were reconstituted with 2.5-5×10⁶ T cell-depleted bone marrow cells of each relevant donor strain at the indicated ratio. Neomycin (50 mg/ml) was added to the drinking water for the next 4 weeks. Chimeras were used for subsequent experiments 6 to 10 weeks after the reconstitution. Percentage of chimerism was tested during the course of the experiments.

### Murine DC Isolation, Analysis and Culture

DC purification from lungs and spleen, analytical and preparative flow cytometry and DC cultures in vitro were performed as described (Wakim et al., 2015 [64]) and see Table 3. The following conjugated monoclonal antibodies were used: anti-CD11c (N418, made in-house), anti-CD4 (GK1.5; made in-house), anti-CD8a (53-6.7; eBioscience), anti-CD11b (M1/70, in house-made), anti-CD24 (M1/69, made in house), anti-CD172a (Sirp-α, made in-house), anti-MHCII (M5/114, made in-house), anti-CD86 (P03, Biolegend), anti-CD45.1 (A20.1; eBioscience), anti-CD103 (2E7; eBioscience), anti-F4/80 (F4/80, made in-house), anti-Latency Associated Protein/TGFβ1 (TW7-16B4, eBioscience), anti-TCR VαD (620.1; made in-house), anti-IL6 (MP5-20F3, BD Pharmingen), anti-IL12 (C15.6, BD Pharmingen), anti TNFα (MP6-XT22, BD Pharmingen), anti-IFNα (XMG1.2, BD Pharmingen), anti-FoxP3 (FJK-16s, eBioscience), anti-IRF4 (3E4, Invitrogen), Fixable Viability Dye (eBioscience). Samples were acquired on LSR-Fortessa or LSR-II (Becton Dickinson) and analyzed using Flowjo Software (TreeStar Inc, Ashland, Oreg.). For cell culture or analysis of RNA, macrophages and DC obtained from pooled lungs of 4-5 mice and sorted with a FACSAria III (purity>95%).

### Human DC and Treg Cells Isolation and Analysis

Circulating DC and monocytes were identified with the following anti-human antibodies: from Biolegend lineage (anti-CD3 (HIT3a), anti-CD14 (63-D3), anti-CD19 (HIB19), anti-CD20 (2H7), anti-CD56 (HCD56)), anti-CD1c (L161), anti-CD11c (3.9), anti-HLA-DR (L243), anti-CD123 (6H6); from BD Biosciences anti-CD141 (1A4) and anti-Blimp-1 (6D3).

Treg cells were identified with CD45-PerCP (clone 2D1), CD25-PC7 (clone 2A3) and CD3-FITC (clone SK7), all from BD Biosciences, and CD127-PE (clone R34.34), CD4-APC (clone 13B8.2) from Beckman Coulter. Treg were identified as CD45⁺CD3⁺CD4⁺CD25^{high}CD127^{low/-}. The number of Treg were deduced from the CD4 T cells number multiplied by the proportion of regulatory T cells in CD4 cells.

### Intracellular Staining of DC and Lymphocytes for Cytokines

For intracellular staining of cytokines in DC or lymphocytes, cell suspensions were obtained by mechanical and collagenase digestion of lungs collected 16 hours after injection of E. coli. Cells were cultured 4 hours in complete media with Golgi Plug, washed twice and then stained for surface markers. Fixation and permeabilization was performed following manufacturer instructions (BD Cytofix/Cytoperm kit, BD Bioscience). Anti-cytokine antibody was incubated overnight at 4° C. Cells were washed twice before FACS analysis.

### Real-Time PCR

RNA was extracted with an RNeasy kit (Qiagen, Valencia, Calif.) from alveolar and interstitial macrophages, CD103⁺ DC and CD11b⁺ DC cells isolated by flow cytometry from the lungs of uninfected mice or from mice 7 days after E. coli infection. Reverse transcription—PCR was performed with a SuperScript III First-strand synthesis system according to manufacturer's instructions (Invitrogen). Real-time PCR was performed with either RT² qPCR Primer sets (Qiagen) specific for mouse TGF-β(UniGene Mm.18213), PLAT (UniGene Mm.154660), aldh1a2 (UniGene Mm.42016), Itgb6 (UniGene Mm.98193) and Itgb8 (UniGene Mm.217000) or primers specific for GAPDH (5′-CCAGGTTGTCTCCTGCGACTT-3′ (SEQ ID NO 3) and 5′-CCTGTTGCTGTAGCCGTATTCA-3′ (SEQ ID NO 4)) and LightCycler 480 SYBR Green I master kit according to the supplier's recommendations (Roche). Relative gene expression was calculated by the 2^{-ΔΔ} Ct method using samples from S group as calibrator.

Measure of mRNA levels of IL12 in conventional dendritic cells during S. aureus pneumonia in naive mice (primary pneumonia, 1ary PN) or in trauma-hemorrhage mice (secondary pneumonia, 2ary PN) were carried out according the method disclosed in Roquilly et al. Eur Resp J 2013, p1365-1378 [46]. In particular, Real-time quantitative PCR was performed on CD11c cells sorted with a CD11c cell isolation kit II (Miltenyi Biotec, Paris, France). These procedures routinely yielded cell populations with purity up to 95%. Total RNA was isolated from sorted cells with TRIzol reagent (Invitrogen, Cergy Pontoise, France) and treated for 45 min at 37 uC with 2 U of RQ1 DNase (Promega, Lyon, France). RNA (1 mg) was reverse-transcribed with superscript III reverse transcriptase (Invitrogen). The cDNA (1 mL) was subjected to RT-qPCR in a BioRad iCycler iQ system using the QuantiTect SYBR Green PCR kit (Qiagen, Courtaboeuf, France). GAPDH was used to normalize gene expression. Relative gene expression was calculated by the 2 Ct method using samples from the sham group as calibrator samples.

Frequencies of IL-12⁺ conventional DC upon in vitro stimulation with LPS of peripheral blood mononuclear cells harvested in healthy controls (HC) and in critically ill patients at the indicated time after acute brain-injury were carried out according the method disclosed in Roquilly et al. PLoS one 2013. In particular, for blood Sample Collection: Venous blood samples were collected in EDTA and heparin vacutainers and processed for analysis within 4 hours on days 2, 5 and 10 after brain-injury. Patient sera were frozen at 280° C. Antibodies and Reagents: DCs were identified using color flow cytometry assay. Briefly, whole blood samples were stained with the following antibodies IL-12-efluor450 (eBiosciences, Paris, France) Abs were used to identify intracellular cytokines after stimulation of peripheral blood mononuclear cells with IL-12. Cytokine Production by Dendritic Cells: Heparinated whole blood samples were incubated for 3h30 at 37 uC under 5% CO₂ conditions with IL-12 for peripheral blood mononuclear cells stimulation. GolgiPlug were added during the last 2h30 hours of incubation to inhibit cellular cytokine release. Control conditions included stimulation with medium alone as negative control. Whole blood samples were then incubated with surface mAbs for 15 min, followed by erythrocyte lysis (BD Biosciences). Samples were then fixed, permeabilised with Cytofix/Cytoperm Plus and stained with cytokine-directed mAbs. The percentages of IL12+ dendritic cells was measured by flow cytometry. Data were analyzed with FlowJo.

Survival curves to S. aureus pneumonia induced in naive mice (1ary PN), in trauma-hemorrhage mice (2ary PN), in trauma-hemorrhage mice injected with NK cells treated ex vivo with MPLA (so called 2ary PN+NK(IL12)) or injected with MPLA-treated DCs (producing IL12 and other cytokines so called DC(IL12)) were carried out according the method disclosed in Roquilly et al. Eur Resp J 2013, p1365-1378 [46].

### BrDU Incorporation

Mice were injected intraperitoneally with 1 mg bromodeoxyuridine (BrdU) (Sigma, St Louis, Mo.) at day 5 and at day 6 after pneumonia. At day 7, macrophages and DC were isolated and analyzed as described (Kamath et al., 2002 [31]).

### Antigen-Presentation of OVA In Vivo

E. coli (DH5α) was shacken for 2 hours at 37° C. in a solution of OVA diluted in luria broth medium (10 mg/ml, in house-made) and washed 2 times in saline before calibration (OD₆₀₀=0.6-0.7) and injection.

OT-II T cells were purified from pooled lymph nodes (inguinal, axillary, sacral, cervical and mesenteric) of transgenic Ly5.1⁺ mice by depletion of non-CD4 T cells and were labeled with Cell Trace Violet as described (Vega-Ramos et al. 2014 [68]). T cell preparations were routinely 85-95% pure, as determined by flow cytometry.

For the assessment of the capacity of antigen presentation in the lungs, mice were injected intra-tracheally with calibrated OVA-coated E. coli or 0.5 μg of anti-DEC205-OVA (clone NLDC-45)(Lahoud et al., 2011 [36]). 1-2.5×10⁶ Violet Cell Tracer-labeled OT-II cells were concomitantly intravenously injected. For the assessment of the capacity of antigen presentation in the spleen, mice were injected i.v. with soluble OVA (0.1 mg) and labeled OT-II cells (1-2.5×10⁶ cells). 60 hours later, cells from the mediastinal lymph node or from the spleen were stained with anti-CD4, CD45.1, anti-TCRVα2 and PI, and resuspended in buffer containing 1-3×10⁴ blank calibration particles (Becton Dickinson). The total number of live dividing OT-II was calculated from the number of dividing cells relative to the number of beads present in each sample.

### Interleukin 12, Anti-TGF-β Monoclonal Antibody and TGF-β Treatments

Mice were treated with IL-12 (100 ng i.p., Abcam) concomitantly to the induction of the secondary pneumonia. Anti-TGFβ monoclonal antibody (1B11, 44 μg i.p. every 3 days) or isotype control IgG1 monoclonal antibody (MG1-45, Biolegend) injections were performed 3 and 6 days after primary pneumonia. TGF-β (1 μg i.p., Thermofisher) was injected once 6 days after primary pneumonia in DT-treated CD11c-DTR chimeric mice

### Quantification and Statistical Analysis

Data were plotted using GraphPad prism (La jolla, CA. United States). Unpaired T-test and Mann-Whitney unpaired test with two-tailed p-values and 95% confidence intervals. One-way ANOVA with Bonferonni correction (post-hoc tests) were used for multiple comparisons. Correlations were investigated by a linear regression test. Correlation between trauma severity (as assessed with Glasgow Coma Scale) and Blimp-1 expression of CD1c DC, or increase of Treg (Delta of Treg Day 7-Day 1) was investigated with a Person test. Statistical details of experiments (exact number of mice per group, exact P-values, dispersion and precision measures) can be found in the figure legends. P<0.05 for statistical significance.

### Results

Recovery from Primary Pneumonia is Followed by Increased Susceptibility to Secondary Infection

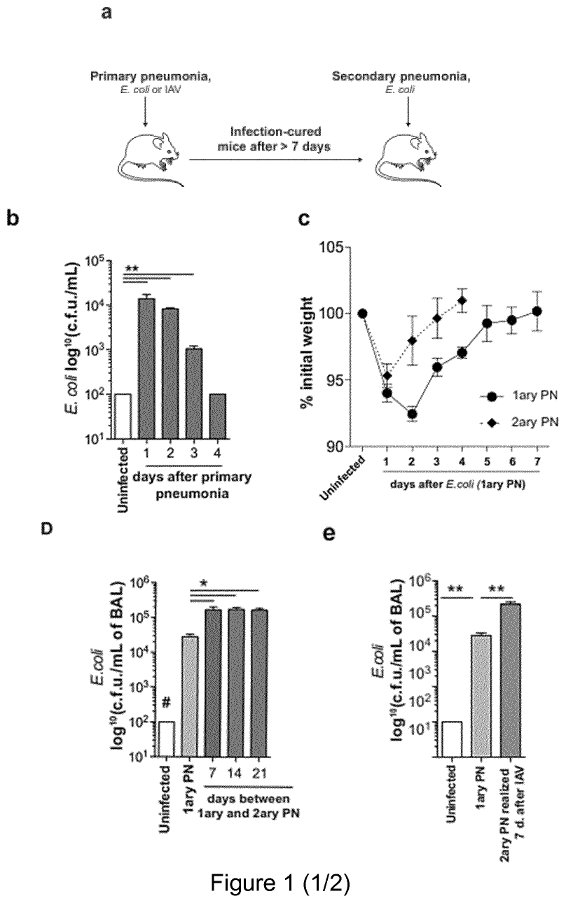

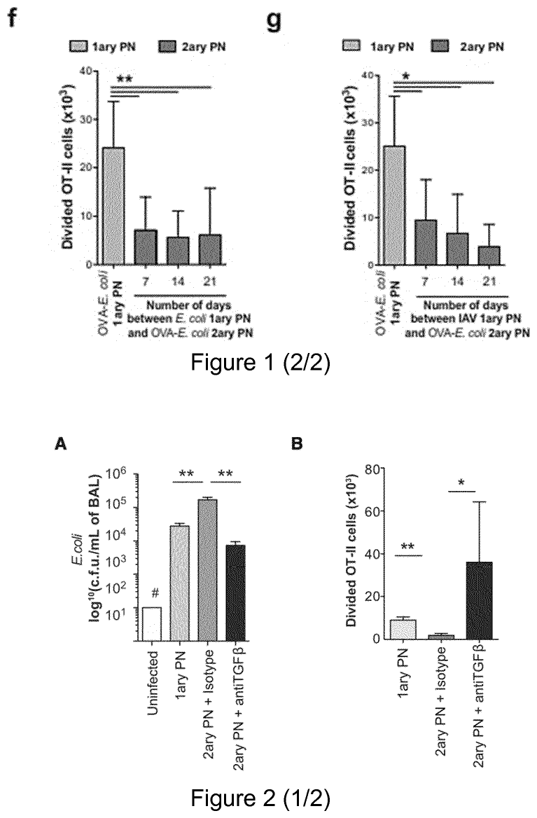

Escherichia coli (E. coli) is the second most frequent gram negative bacilli involved in both community- and hospital-acquired pneumonia (Roquilly et al., 2016 [47]; van Vught et al., 2016b [60]). Early recurrence of pneumonia to the same pathogens is observed in up to 20% of critically ill patients cured from primary pneumonia (Chastre et al., 2003). To mimic in mice this clinical scenario, we induced secondary pneumonia with E. coli in mice cured from a bacterial (E. coli) or a viral (influenza A virus, IAV) primary pneumonia (FIG. 1a). During primary E. coli pneumonia, pathogen burden and mouse weight loss peaked 1 day after infection and then decreased until by day 7 the mice had cleared the bacterium (FIG. 1b) and recovered their normal weight (FIG. 1c). If these mice were re-infected with E. coli 7 to 21 days after the primary infection, they suffered more severe (secondary) pneumonia with increased bacterial burden and weight loss (FIG. 1 c,d). Similarly, mice infected with E. coli suffered more severe pneumonia if they had previously been infected with, and recovered from primary pneumonia caused by IAV (Wakim et al., 2013 [63]; 2015 [64]).

Defective CD4 T cell priming following recovery from primary pneumonia

T cell priming in mice that recovered from bacterial pneumonia by re-infecting them 7-21 days after the primary infection with E. coli associated with the model antigen, ovalbumin (OVA) was assessed. The mice also received MHC II-restricted, OVA-specific OT-II cells, which proliferated in the mediastinal lymph nodes (LN) in response to local presentation of OVA. A severe reduction in OT-II proliferation during secondary pneumonia compared to that observed in mice that received E. coli-OVA as a primary infection (FIG. 1f) was observed. Similar results were obtained in mice where primary pneumonia was caused by IAV (FIG. 1g).

### TGF-β is Involved in Pneumonia-Induced Immunosuppression Via Treg Cell Induction

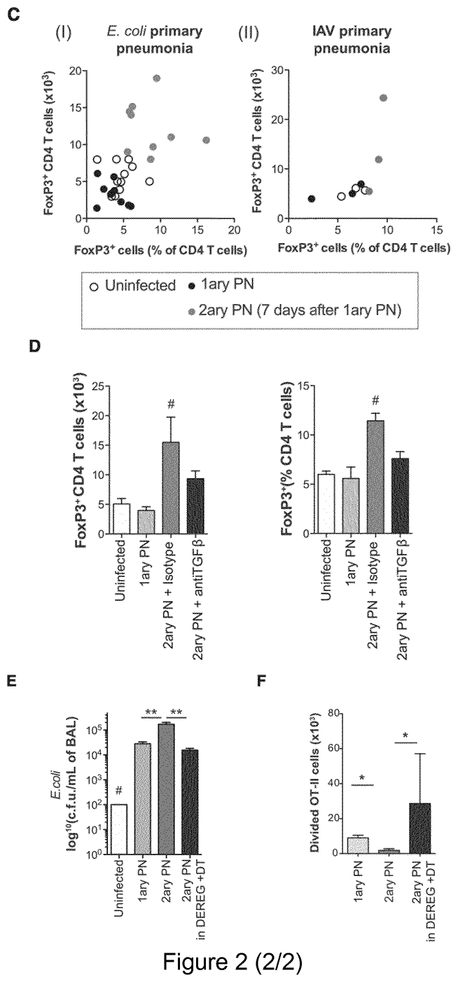

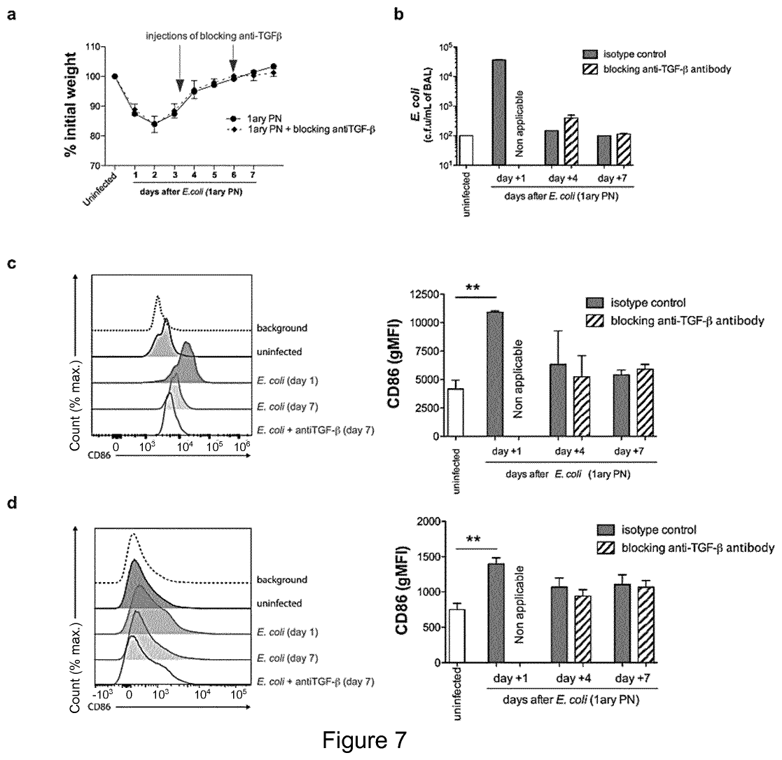

Tumor growth factor (TGF)-β is critical for tissue healing after injury and is immunosuppressive (Akhurst and Hata, 2012 [1]). To test whether TGF-β released within lung tissue during or after primary pneumonia induced immunosuppression, TGF-β released was neutralized with a mAb injected 3 and 6 days after initiation of primary pneumonia. This treatment did not affect bacterial burden or weight changes during primary infection (FIG. 7 a-d), but it caused reduced bacterial burden and increased OT-II cell priming during secondary pneumonia (FIG. 2a,b). This indicated a role for TGF-β on the induction of immunosuppression after recovery from primary infection.

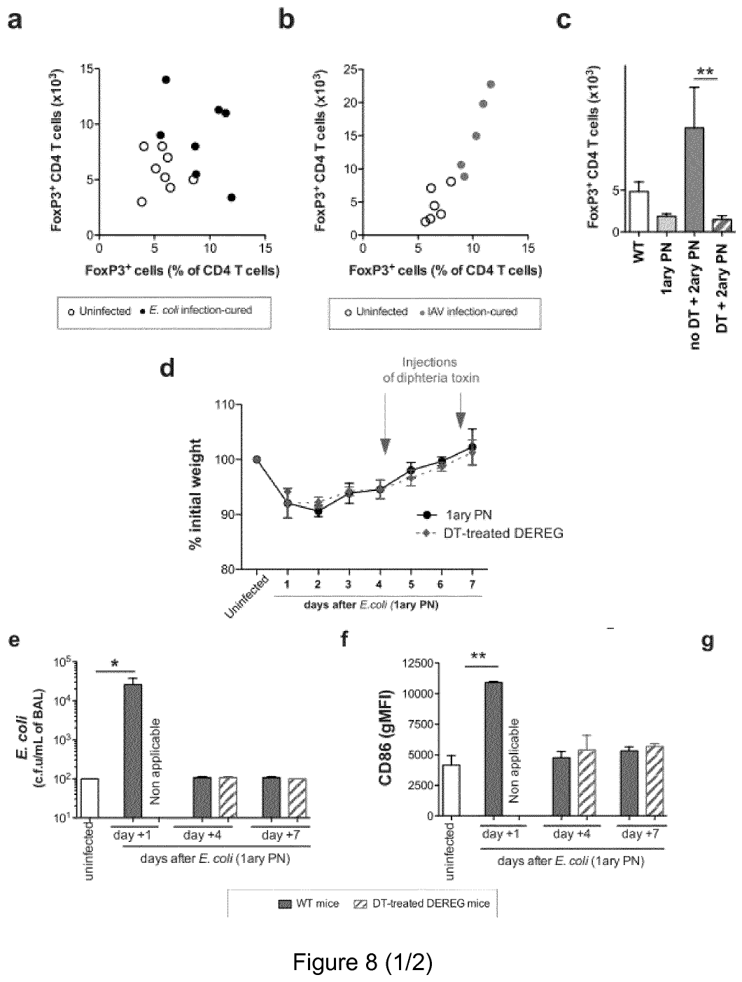

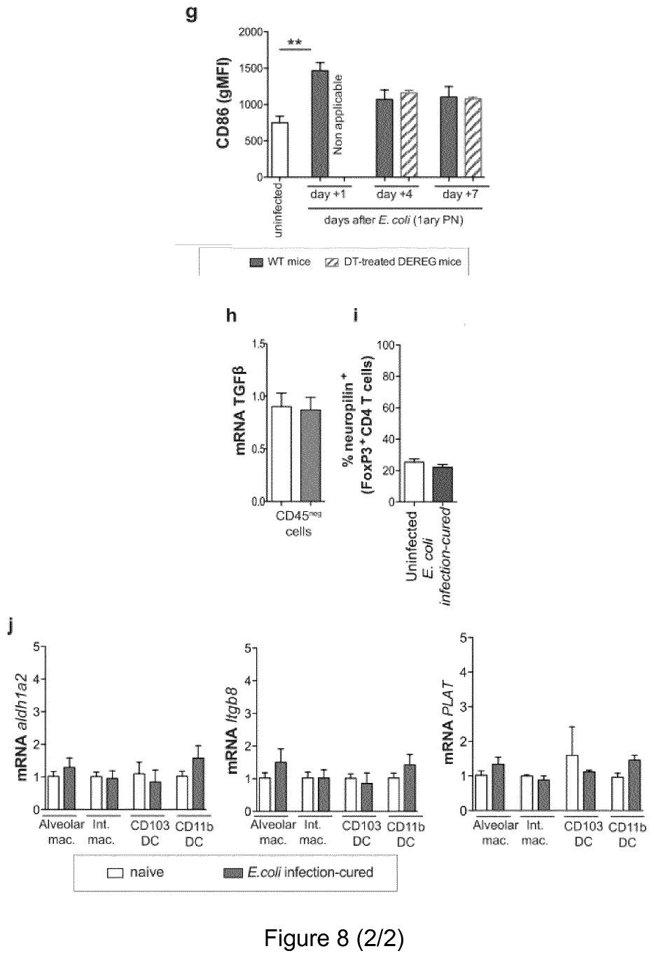

TGF-β induces differentiation of naive CD4 T cells into FoxP3⁺ T regulatory (Treg) cells (Chen et al., 2003 [18]). The inventors demonstrate a higher proportion of lung Treg cells after recovery from primary bacterial or IAV pneumonia (FIG. 8a-b), and also in the lungs of mice suffering secondary pneumonia (FIG. 2c), than in mice uninfected or suffering primary pneumonia. Treatment with anti-TGF-β reduced Treg cells accumulation (FIG. 2d), so the role of Treg cells in susceptibility to secondary infection was investigated. transgenic mice expressing the diphtheria toxin receptor (DTR) in FoxP3⁺ cells (DEREG mice) were infected, where the inventors could deplete Treg cells after initiation of primary or secondary pneumonia (FIG. 8c). Depletion of Treg cells during the resolution of primary pneumonia (from days 4 to 7 post primary infection) did not alter the course of this infection (FIG. 8d-e), but restored the effectiveness of bacterial clearance, and enhanced CD4⁺ T cell priming, during secondary pneumonia (FIG. 2e,f). Thus, TGF-β in the lungs of mice that recovered from primary pneumonia induced Treg cell accumulation during secondary pneumonia, which contributed to immunosuppression.

### Macrophages and DC Produce TGF-β in Infection-Cured Mice

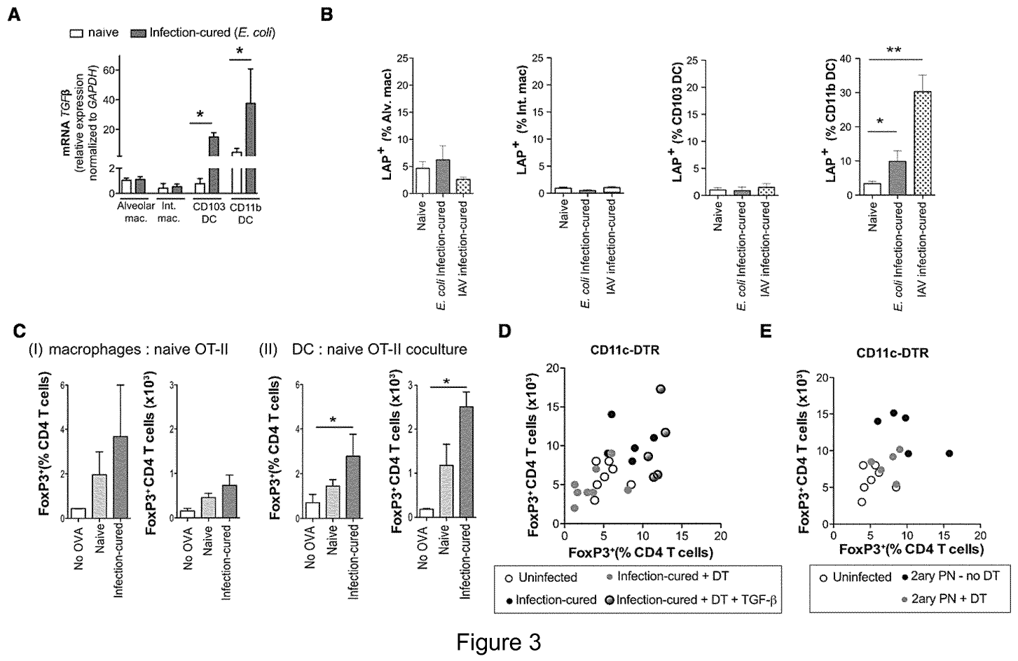

The cells that produced TGF-β in the lungs of mice cured from primary infection were next identified. Expression of TGF-β mRNA did not vary in non-hematopoietic cells (CD45^{neg}) in infection-cured mice (FIG. 8h), suggesting the cells responsible were hematopoietic. Macrophages and DC produce and activate TGF-β, inducing Treg cell formation (Chen et al., 2003), and the Treg cells that accumulated in infection-cured mice were neuropilin^{neg} (FIG. 8i), indicating they were peripherally induced rather than thymus-derived, natural Treg (Weiss et al., 2012 [65]). An increased TGF-β mRNA expression in lung DC of mice recovered from primary pneumonia (FIG. 3a), although expression of RNA for TGF-β activators remained unchanged (FIG. 8j), was demonstrate. Production of TGF-β protein, as assessed by the membrane expression of its inactive precursor Latency-Associated Peptide (LAP) (Annes et al., 2003 [3]), was increased in CD11b⁺ DC of mice cured from primary pneumonia compared to DC of naive mice (FIG. 3b). This correlated with the ability of these DC to induce Treg cells in vitro (FIG. 3c). We used CD11c-DTR transgenic mice, where we could deplete both macrophages and DC (FIG. 9a-b), to test their role in Treg cell induction. Their depletion did not affect the number of lung CD4 T cells (FIG. 9c), but it reduced the number of Treg cells in mice recovered from primary pneumonia (FIG. 3d) or suffering secondary pneumonia (FIG. 3e). This reduction was reversed with anti-TGF-β treatment (FIG. 3d, red dots). Altogether, these results indicate that DC and macrophages of mice that recovered from primary pneumonia produce TGF-β and promote Treg cell differentiation.

Macrophages and DC Newly Produced after Severe Primary Pneumonia are Paralyzed

DC and macrophages become activated, increase in numbers (FIG. 9d-e) and elicit protective immunity (FIG. 9f-g) against primary E. coli infection, yet as shown above these cells appear critical in the induction of tolerance to secondary infection. A comparison of the function and phenotype of these two cell types before, during and after primary pneumonia was carried out.

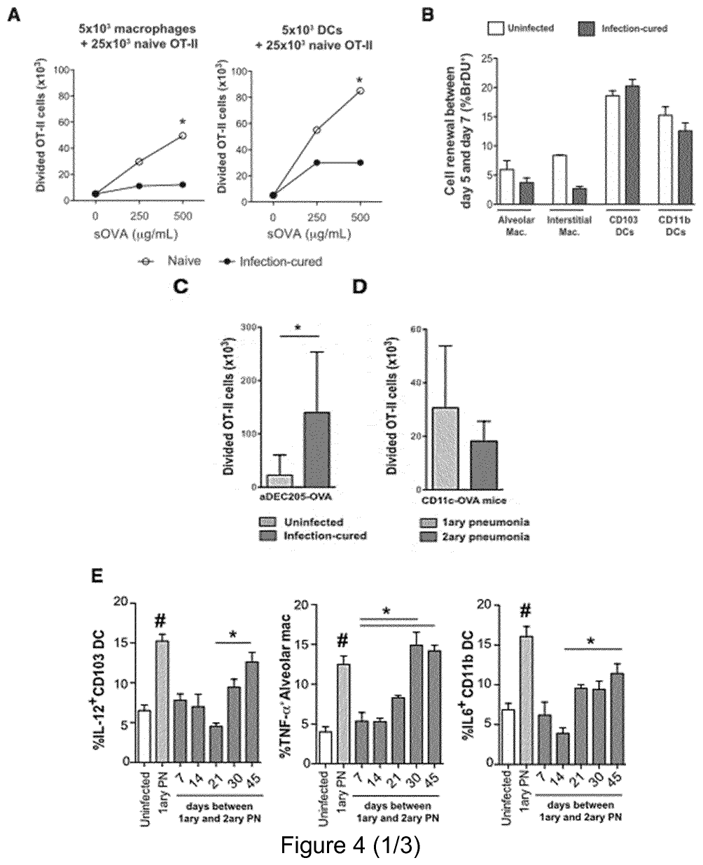

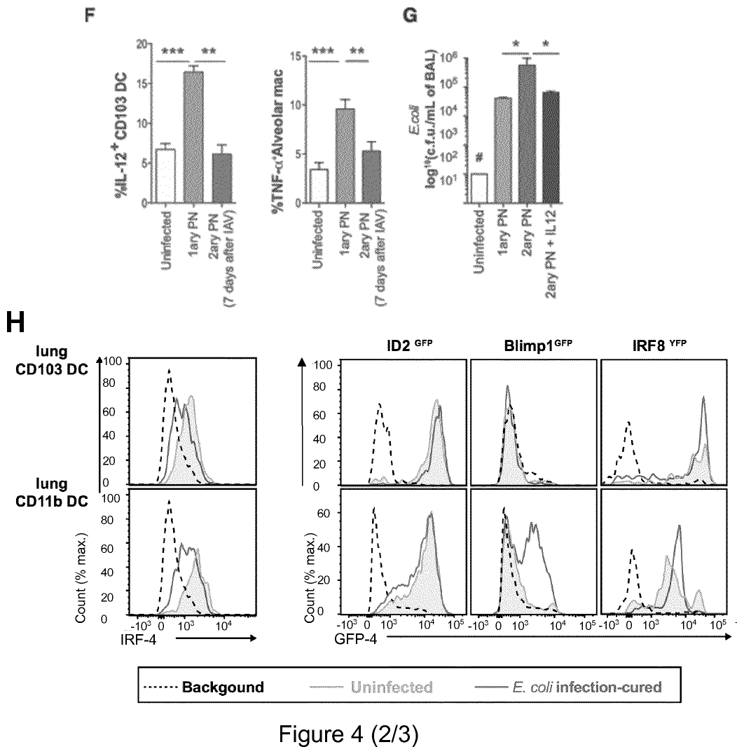

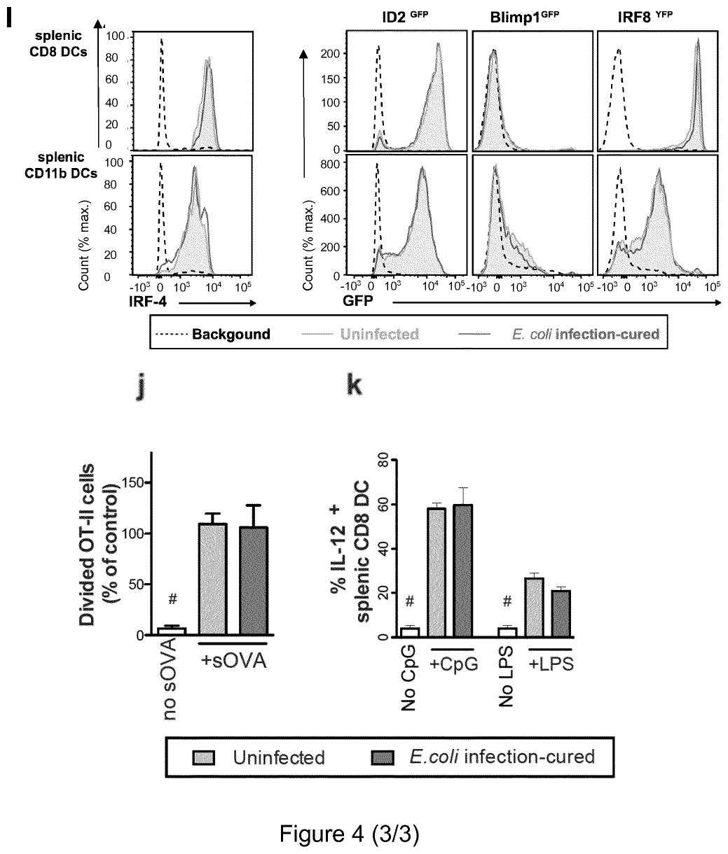

Capture and presentation of pathogen antigens via MHC-II is a hallmark property of DC and macrophages (Guilliams et al., 2013 [23]; Segura and Villadangos, 2009 [53]), and though their numbers during primary and secondary pneumonia were comparable (FIG. 10a), MHC II-mediated T cell priming was defective in mice suffering secondary pneumonia for at least 21 days after recovery from primary infection (FIG. 1e,f). Both macrophages and DC of mice that recovered from primary pneumonia showed defective antigen presentation capacity in vitro (FIG. 4a). the inventors have demonstrated that primary pneumonia causes systemic activation of lung DC (FIG. 10b), and since mature DC cannot present newly encountered antigens (Vega-Ramos et al., 2014 [62]; Wilson et al., 2006 [66]), persistent DC maturation might explain the lack of antigen presentation by DC in mice that recovered from primary pneumonia. However, neither DC nor macrophages exhibited signs of activation (high CD86 expression) at that stage (FIG. 10b). Moreover, measurements of BrDU incorporation showed the rate of macrophages and DC renewal in mice recovered from primary pneumonia was comparable to that in non-infected mice (FIG. 4b). This suggested that, as the mice recovered from primary pneumonia, activated DC and macrophages were replaced by “immature” cells that were defective at detecting and/or presenting antigen from a secondarily infecting pathogen. Both DC and macrophages increased CD86 expression at the onset of secondary infection with E. coli in the lungs (FIG. 10c-d), demonstrating they were still responsive to pathogens. Targeting antigen to a surface DC receptor can overcome defects in antigen presentation (Lahoud et al., 2011 [36]), and we observed effective OT-II priming in mice recovered from primary pneumonia if they received OVA conjugated to a mAb that recognizes the DC receptor, DEC-205 (FIG. 4c). Furthermore, OT-II priming occurred in infection-cured CD11c-OVA transgenic mice challenged with a secondary E. coli infection, in which macrophages and DC constitutively express and present OVA (FIG. 4d). Therefore, OT-II activation and induction of proliferation can occur in mice suffering a secondary infection if the T cells encounter their cognate MHC-peptide complex on the surface of CD11c^{high} cells (DC). This series of experiments demonstrate that DC and macrophages, which continually turn-over in the lungs (Kamath et al., 2002), develop with impaired capacity to capture, process and/or generate MHC-II-peptide complexes for 21 days or more after recovery from primary pneumonia.

### Cytokine Production by Macrophages and DC During Secondary Pneumonia

Production of immunogenic cytokines by macrophages and DC is as critical, if not more, for the control of infection than antigen presentation, as these cytokines regulate both innate and T cell-dependent immunity (Marchingo et al., 2014 [40]). We identified CD103⁺ DC, alveolar macrophages and CD11b⁺ DC as the main sources of interleukin (IL)-12, tumor necrosis factor (TNF)-β and IL-6, respectively, during E. coli primary pneumonia (FIG. 11a). Production of these cytokines during secondary pulmonary infection with E. coli was significantly impaired for up to 30 days in mice recovered from primary E. coli or IAV pneumonia (FIG. 4e,f). This defect was apparent even if the dose of E. coli causing secondary pneumonia was increased 3.3 times (FIG. 11b), or if the bacterium causing the secondary pneumonia was different to the one that caused the primary one (e.g. Staphylococcus aureus or Pseudomonas aeruginosa, FIG. 11c). IL-12 is required to elicit interferon (IFN)-γ production by NK cells (FIG. 11d), a cytokine that in turn plays a critical role in the resolution of bacteria-induced pneumonia (Broquet et al., 2014 [10]). Treatment of mice with IL-12 during secondary pneumonia enhanced bacterial clearance (FIG. 4g), and restored IFN-γ production by NK cells (FIG. 11e). These results show that defective immunogenic cytokine production by macrophages and DC plays a central role in the increased susceptibility of infected-cured mice to secondary pneumonia.

In other words, as (IFN)-γ is a marker the induced immunosuppression. The example clearly demonstrate that IL-12 restores (IFN)-γ production and thus allows to treat immunosuppression. Accordingly, this example clearly demonstrates that the present invention allows to prevent and/or treat of secondary infection, in particular by suppressing the primary infection induced immunosuppression.

### Altered Transcriptional Programming in DC Following Primary Pneumonia

A comparison of the expression of phenotypic markers and immunoregulatory factors in DC before and after pneumonia was carried out. The inventors demonstrated no significant changes in the expression of characteristic surface markers of DC (CD11c, CD24, MHC-II, DEC205, CD103 and CD11b) and macrophages (F4/80, CD64, Ly6G, CD11c, CD11b) (FIG. 12a-b). In contrast, the amounts of key transcription factors involved in the control of immunogenic vs tolerogenic functions of DCs were significantly altered (Steinman et al., 2003 [55]) (FIG. 4h). Specifically, the amount of IRF4, which promotes antigen presentation to CD4 T cells (Vander Lugt et al., 2013 [61]), was lower in CD11b+DC and in CD103⁺ DC after clearance of the infection (FIG. 4h). Conversely, expression of the transcription factor Blimp1, which induces tolerogenic functions in DC (Kim et al., 2011 [33]), was increased (FIG. 4h). Expression of two other transcription factors involved in DC development and whose expression in DC is critical for immune response to pathogens (Belz and Nutt, 2012 [8]; Hambleton et al., 2011[24]), ID2 and IRF8, remained unchanged (FIG. 4h). Expression of these four transcription factors did not change in macrophages (FIG. 12c). Thus, although neither the rate of DC turn-over (FIG. 4b) nor the expression of characteristic surface markers of DC subtypes changed substantially after resolution of primary pneumonia, the expression of transcription factors that regulate DC function did.

### DC Programming is Locally Mediated by Secondary Inflammatory Signals

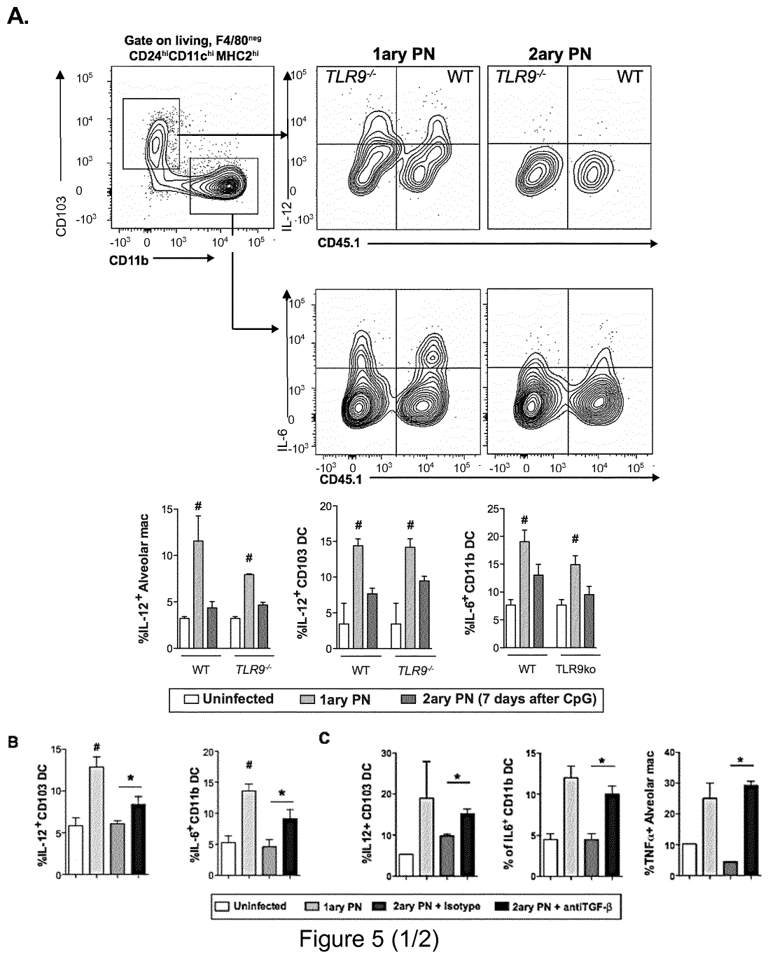

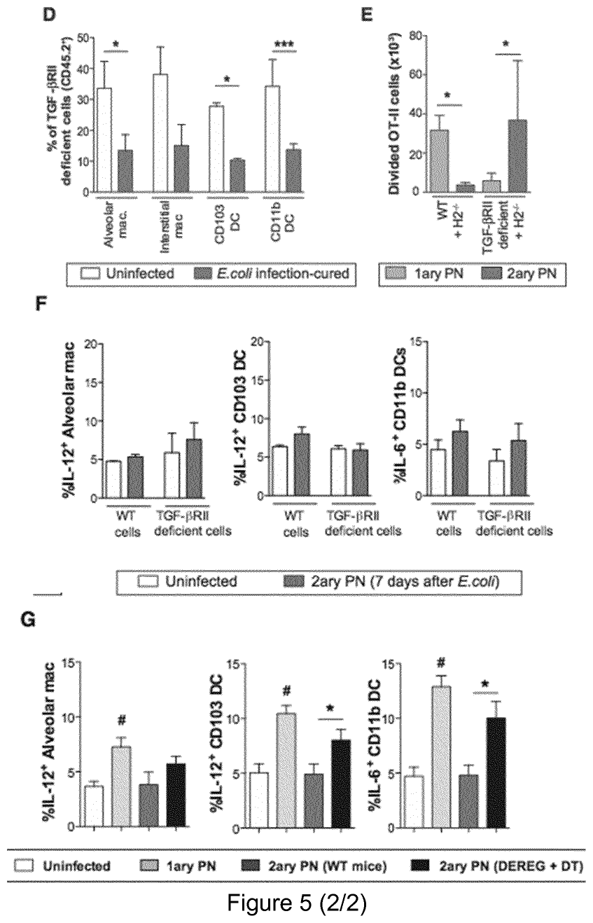

To examine whether the signals responsible for reprogramming of DC after pneumonia acted systemically, the phenotype and function of splenic DC 7 days following primary pneumonia were assessed. Neither the expression of transcription factors (FIG. 4i), nor capacity for T cell priming and cytokine production of these cells were altered 7 days after E. coli pneumonia (FIG. 4j,k). Therefore, signals that induce altered DC functions after recovery from primary pneumonia only act locally on cells that undergo terminal differentiation in the same tissues that suffered the infection. Next we addressed whether such signals consisted of pathogen-derived products that lingered at the infection site (Cegelski et al., 2008 [14]), or of endogenous mediators produced by the affected tissues (Vega-Ramos et al., 2014 [62]). A mixed-bone marrow chimeras where wild-type (WT) mice received a 1:1 ratio of bone marrow from WT (CD45.1⁺) or Tlr9^{-/-} (CD45.1⁻) donors (FIG. 13a) were generated. In this setting, Tlr9^{-/-} cells cannot recognize the pathogen-associated molecular pattern mimic CpG, but can receive signals from secondary mediators released by WT cells responding to CpG (Vega-Ramos et al., 2014 [62]). Intra-tracheal administration of CpG induced a lung inflammatory response that caused lung DC and macrophage activation, followed by a 7-day long recovery phase in which the activated cells were replaced by immature cells (FIG. 13b-c), reproducing the time course of the recovery from E. coli or IAV infection. At day 7 post-CpG treatment, the chimeric animals were challenged with E. coli and measured cytokine production by WT and Tlr9^{-/-} DC or macrophages, finding both groups of cells displayed reduced production of IL-12 and IL-6 compared to their counterparts from naïve mice (FIG. 5a). This result implied and demonstrate that the functional alterations observed in DC and macrophages following recovery from pneumonia were induced not by direct encounter of pathogen products but by secondary mediators of inflammation.

TGF-β Contributes to Program Macrophages and DC after Resolution of Primary Pneumonia

Neutralization of TGF-β with a blocking mAb injected during, or after resolution of, primary pneumonia reduced the defects in DC cytokine production during secondary infection (FIG. 5b-c). To explore further the role of TGF-β signaling on DC modulation, Tgfbr2^{fl/fl}Cd11c^{cre} mice were used, which lack expression of TGF-β receptor selectively in DC and macrophages. These mice spontaneously succumb to inflammatory disease (Ramalingam et al., 2012), mixed bone marrow chimeras where WT mice were reconstituted with a 1:3 mix of Tgfbr2^{fl/fl}Cd11c^{cre} and WT bone marrow were generated. Seven days after E. coli infection, the proportion of TGF-βR-deficient CD11c cells (CD45.2⁺ cells) in the lungs of these mice was significantly lower than in uninfected chimeras (FIG. 5d), confirming the role of TGF-β in macrophage and DC renewal after infection.

In order to investigate the role of TGF-β signaling in macrophages and DC on their ability to prime CD4 T cells, mixed bone marrow chimeras where WT mice were reconstituted with a 1:3 mix of Tgfbr2^{fl/fl}Cd11c^{cre} and H2^{-/-} (MHC-II-deficient) bone marrow were generated. In these chimeric mice, only the cells derived from the Tgfbr2^{fl/fl}Cd11c^{cre} bone marrow are able to present antigen to, and prime, CD4 T cells. TGF-βR-deficient CD11c cells retained the ability to elicit effective priming of OT-II cells during secondary pneumonia in vivo (FIG. 5e).

Finally, reduced cytokine production by DC and macrophages during secondary pneumonia was examined to know whether it was also caused by TGF-β recognition by the cells themselves. This did not appear to be the case because in WT:Tgfbr2^{fl/fl}Cd11c^{cre} mixed bone marrow chimeras both the WT and the TGF-βR-deficient cells were impaired during secondary pneumonia (FIG. 5f). Since neutralization of TGF-β rescued IL-12 and IL-6 production by DC and macrophages (FIG. 5b-c), TGF-β act through an indirect mechanism to impair cytokine production by the two cell types. Treg cells are known to inhibit DC functions (Onishi et al., 2008 [43]) and depletion of Treg cells during the resolution of primary pneumonia enhanced IL-12 and IL-6 production by macrophages and DC during secondary infection (FIG. 5g). Together, our results indicate a pivotal role for TGF-β in the induction of DC and macrophages with reduced immunogenic function. It acts directly on developing cells, and indirectly via Treg cells.

### Expression of Blimp1 in CD1c⁺ DC and Numbers of Treg Cells in Human Patients Suffering Systemic Inflammatory Response Syndrome

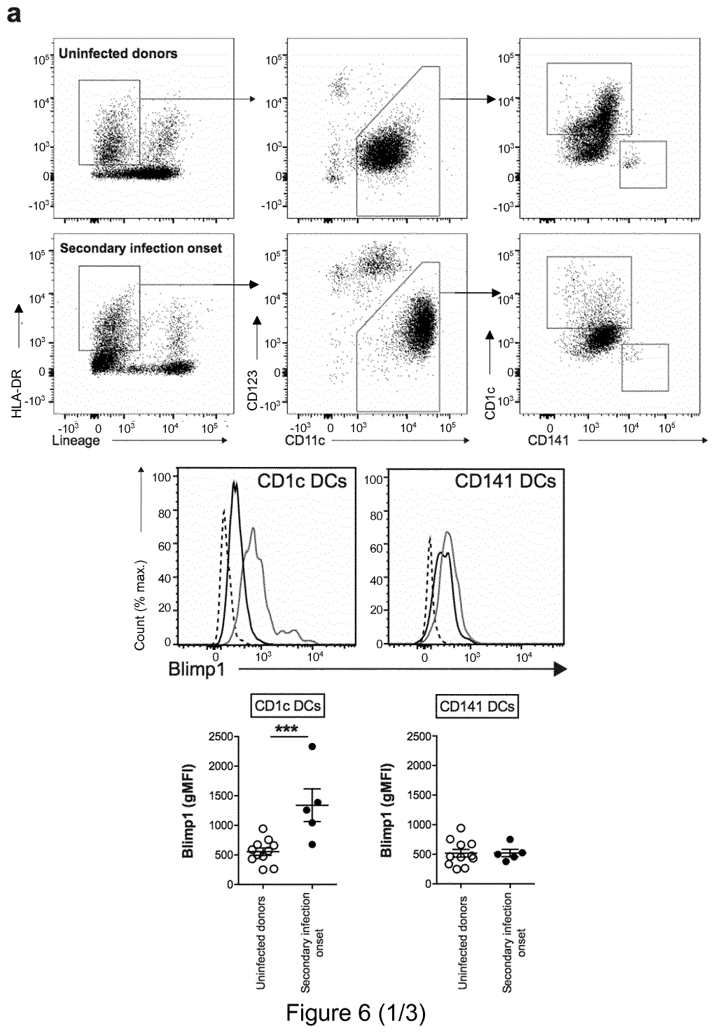

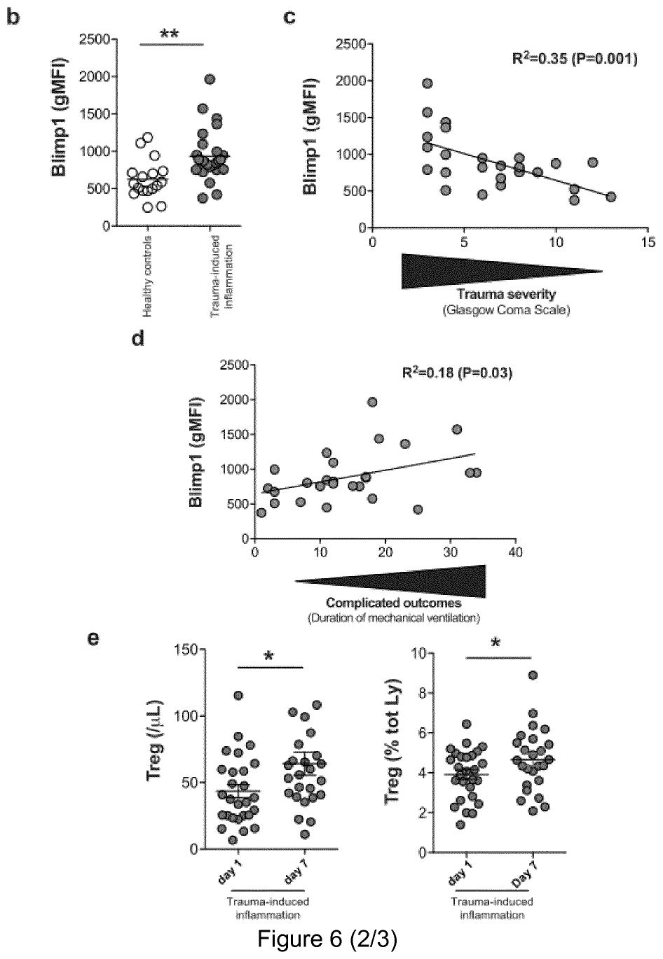

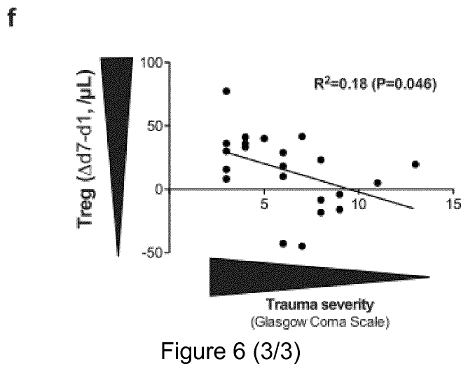

First, PBMC from a prospective cohort of septic patients presenting with E. coli secondary infection [IBIS sepsis, n=5 (Table 1)] was analyzed. Human CD1c DC, the circulating equivalent of murine CD11b DC (Guilliams et al., 2014 [23]), expressed high level of the transcription factor Blimp1 in these patients as compared to matched uninfected donors (FIG. 6a), paralleling the result observed in mice. Also reproducing what observed in infected-cured mice (FIG. 4h), CD141 DC (the human equivalent of murine CD103⁺ DC) did not show increased Blimp1 expression in these patients (FIG. 6a).

TABLE 1
Characteristics of the SIRS sepsis population

| | Healthy controls | | Septic patients | |
|---|---|---|---|---|
| | (n = 12) | | (n = 5) | |
| Age (years) | 55 | (45-53) | 65 | (62-73) |
| Sex ratio (Male/Female) | 22/10 | (69) | 4/1 | (80) |
| Severity scores on | | | | |
| admission | | | | |
| SAPS-II | NA | | 73 | (72-80) |
| SOFA | NA | | 11 | (11-11) |
| Signs of Systemic | | | | |
| inflammatory response | | | | |
| Syndrome on admission | | | | |
| Abnormal heart rate | NA | | 5 | (100) |
| Abnormal body temperature | NA | | 5 | (100) |
| (>39.9) | | | | |
| Abnormal respiratory rates | NA | | 5 | (100) |
| Abnormal white-cell count | NA | | 5 | (100) |
| White cell count (G/L) | NA | | 14500 | (12300-18900) |
| Localisation of the primary | | | | |
| infection | | | | |
| Abdomen | NA | | 3 | (60) |
| Lungs | NA | | 2 | (40) |
| Hospital acquired infection | | | | |
| Pneumonia | NA | | 3 | (60) |
| Other side (abdomen, urine) | | | 2 | (40) |
| Escherichia coli, yes | | | 5 | (100) |

| | | | |
|---|---|---|---|
| Duration of invasive | NA | 8 | (5-15) |
| ventilation (days) | | | |
| Duration of intensive Care | NA | 13 | (10-21) |
| unit Hospitalization (days) | | | |
| Death at day 90 | NA | 1 | (20) |

In table 1 results are expression as N(%) or median (25-75 percentile)
NA: not applicable,
SAPS-II: simplified acute physiology score,
SOFA: sequential organ failure assessment

The inventors demonstrate that in mice that the reprogramming of DC is not pathogen-driven but induced by secondary mediators of inflammation, the inventors demonstrate that Blimp1⁺ CD1c DC are also be observed in patients suffering aseptic systemic inflammatory response syndrome (SIRS). Circulating DC from patients suffering from trauma-induced severe inflammation [IBIS cohorts 1 and 2, n-32 and n-29 respectively Table 2] was examined. Circulating DC from these patients have lost their ability to produce TNF-α, IL-6 and IL-12 upon in vitro stimulation reproducing a hallmark of mouse paralyzed DC (FIG. 4e,f). Blimp1 expression was also increased in circulating CD1c DC collected from these trauma patients as compared to matched healthy controls (FIG. 6b). The level of expression of Blimp1 in circulating CD1c DC increased with the severity of the trauma (FIG. 6c) and correlated with the duration of mechanical ventilation, a surrogate marker of complicated outcome (FIG. 6d). The inventors also demonstrate an increase in the number and frequency of circulating Treg cells in trauma patients (FIG. 6e), which again correlated with trauma severity (FIG. 6f).

TABLE 2
Definitions of macrophages and dendritic cells.

| | Alveolar | Interstitial | Conventional | Conventional |
|---|---|---|---|---|
| | macrophages | macrophages | CD103 DCs | CD11b DCs |
| CD24 | −/+ | −/+ | +++ | +++ |
| F4/80 | ++ | ++ | − | − |
| MHC-II | − | ++ | +++ | +++ |
| CD11c | +++ | + | +++ | +++ |
| CD11b | −/+ | +++ | + | +++ |
| CD103 | − | − | ++ | − |
| A posteriori | | | | |
| analysis | | | | |
| CD64 | ++ | ++ | − | − |
| FcRε1 | ++ | + | − | − |
| (MAR-1) | | | | |

### DISCUSSION

The effector mechanisms deployed by the immune system to fight pathogens can cause tissue damage and have to be tightly controlled to prevent self-harm. Here the example demonstrate a network of regulatory mechanisms that dampen the immune response locally in response to lung infection. It involves multiple cell types and cytokines, with macrophages and DC playing a pivotal role. Importantly, after clearance of the infection the immunosuppression induced by these mechanisms does not restore immune homeostasis to the situation that preceded the infection. It persists locally for weeks after resolution of the infection, increasing the susceptibility to secondary infections.

The examples demonstrate that treatment with IL-12 or inhibitors of TGF-β allows to restore the immunity of a subject after an infection and also to treat secondary infection and/or nosocomial infection.

DC respond quickly to pathogen encounter by presenting antigens to induce T cell responses, and releasing cytokines that promote both innate and adaptive immunity (Banchereau and Steinman, 1998 [6]). During this response they undergo a process of “maturation” that involves multiple genetic, phenotypic and functional changes (Landmann et al., 2001 [37]; Wilson et al., 2006 [66]). DC have a short half-life, both in the steady-state and after infection (Kamath et al., 2002 [31]), being continually replaced by new DC derived from precursors immigrated from the bone marrow (Geissmann et al., 2010 [21]). Final DC differentiation occurs in peripheral tissues under the influence of local cytokines that modulate the responsiveness and functional properties of the newly produced DC (Amit et al., 2015 [2]). The results demonstrate that the DC that develop in the lung after resolution of pneumonia have diminished capacity to present antigens and to secrete immunostimulatory cytokines, which makes them less capable of initiating adaptive and innate immunity against a secondary bacterial infection.

The invention allow to overcome the deficiency of DC cells, for example diminished capacity to present antigens and to secrete immunostimulatory cytokines, with the administration of IL-12 or inhibitors of TGF-β which allows to restore the immunity of a subject after an infection and thus allow to prevent or to treat secondary infection and/or nosocomial infection.

In addition, the inventors are the first who demonstrate that DC cells produce higher levels of TGF-β, which promotes accumulation of Treg cells. They demonstrate that the signals that promote the differentiation of DC to this paralyzed state are not directly associated with the pathogen that caused the primary infection; they are mediated by secondary cytokines acting locally. They also demonstrate that TGF-β plays a prominent role in the differentiation of paralyzed DC, although our results do not discard a role for other cytokines or surface receptors. The source of active TGF-β may be multiple cell types.

As demonstrated in the example, the invention allow to overcome the deficiency of DC cells, for example diminished capacity to present antigens and to secrete immunostimulatory cytokines, with the administration of IL-12 or inhibitors of TGF-β which allows to restore the immunity of a subject after an infection and thus allow to prevent or to treat secondary infection and/or nosocomial infection.

In addition, the inventors have clearly demonstrates that production of IL-12 by dendritic cells and macrophages is critical for the innate immune response and clinical recovery to bacterial pneumonia, the production of IL-12 by macrophages and dendritic cells is drastically decreased during bacterial pneumonia in mice and in humans cured from a primary infection or after non-septic inflammatory response (such as trauma, brain-injury), and that IL-12 treatment restores innate immune response and enhances clinical recovery during bacterial pneumonia in mice cured from infection or from trauma-hemorrhage.

Accordingly, the inventor have clearly demonstrate that the present invention allows to treat secondary infection and/or nosocomial infection, in particular since the treatment is not directly directed to the pathogen or the cause of the disease but improve the defense of the treated subject.

The effects reported can be considered an extension of the phenomenon of “immunological training” induced locally by commensal flora and other environmental stimuli (Carr et al., 2016 [12]). The long term immunosuppression that ensues in mice or humans that survive severe infections can be considered a deleterious consequence of over-adaptation to a challenge that in normal conditions would lead to death but can be overcome in the controlled conditions of the laboratory (mice) or intensive care units (humans). Importantly, the inventors demonstrate that the signals that cause local cell imprinting are non-antigen specific, explaining why recovery from a primary infection can increase the susceptibility to an entirely new pathogen.

The inventors demonstrate that circulating DC of sepsis or trauma patients express characteristic markers of mouse paralyzed DC such as a high level of Blimp1. The presence of Blimp1^{high} DC in critically-ill patients is a prognostic marker of extended immunosuppression, affording an opportunity for early intervention to prevent secondary infections in this high-risk cohort of patients.

### LIST OF REFERENCES

1. Akhurst, R. J., and Hata, A. (2012). Targeting the TGFβ signalling pathway in disease. Nat. Rev. Drug. Discov. 11, 790-812.
2. Amit, I., Winter, D. R. and Jung, S. (2015). The role of the local environment and epigenetics in shaping macrophage identity and their effect on tissue homeostasis. Nat. Immunol. 17, 18-25.
3. Annes, J. P., Munger, J. S. and Rifkin D. B. (2003). Making sense of latent TGFbeta activation. J. Cell. Science 116, 217-224.
4. Asehnoune, K., Roquilly, A. and Abraham, E. (2012). Innate immune dysfunction in trauma patients: from pathophysiology to treatment. Anesthesiology 117, 411-416.
5. Askenase, M. H., Han, S.-J., Byrd, A. L., Morais da Fonseca, D., Bouladoux, N., Wilhelm, C., Konkel, J. E., Hand, T. W., Lacerda-Queiroz, N., Su, X.-Z., et al. (2015). Bone-Marrow-Resident NK Cells Prime Monocytes for Regulatory Function during Infection. Immunity 42, 1130-1142.
6. Banchereau, J., and Steinman, R. M. (1998). Dendritic cells and the control of immunity. Nature 392, 245-252.
7. Barnden, M. J., Allison, J., Heath, W. R. and Carbone, F. R. (1998). Defective TCR expression in transgenic mice constructed using cDNA-based alpha- and beta-chain genes under the control of heterologous regulatory elements. Immunol. Cell. Biol. 76, 34-40.
8. Belz, G. T., and Nutt, S. L. (2012). Transcriptional programming of the dendritic cell network. Nat. Rev. Immunol. 12, 101-113.
9. Beura, L. K., Hamilton, S. E., Bi, K., Schenkel, J. M., Odumade, O. A., Casey, K. A., Thompson, E. A., Fraser, K. A., Rosato, P. C., Filali-Mouhim, A., Sekaly, R. P., Jenkins, M. K., et al. (2016). Normalizing the environment recapitulates adult human immune traits in laboratory mice. Nature 532, 512-516.
10. Broquet, A., Roquilly, A., Jacqueline, C., Potel, G., Caillon, J. and Asehnoune, K. (2014). Depletion of natural killer cells increases mice susceptibility in a Pseudomonas aeruginosa pneumonia model. Crit. Care. Med. 42, e441-50.
11. Campisi, L., Barbet, G., Ding, Y., Esplugues, E., Flavell, R. A. and Blander, J. M. (2016). Apoptosis in response to microbial infection induces autoreactive TH17 cells. Nat. Immunol. 17, 1084-1092.
12. Carr, E. J., Dooley, J., Garcia-Perez, J. E., Lagou, V., Lee, J. C., Wouters, C., Meyts, I., Goris, A., Boeckxstaens, G., Linterman, M. A., and Liston, A. (2016). The cellular composition of the human immune system is shaped by age and cohabitation. Nat. Immunol. 17, 461-468.
13. Caton, M. L., Smith-Raska, M. R., and Reizis, B. (2007). Notch-RBP-J signaling controls the homeostasis of CD8− dendritic cells in the spleen. J. Exp. Med. 204, 1653-1664.
14. Cegelski, L., Marshall, G. R., Eldridge, G. R., and Hultgren, S. J. (2008). The biology and future prospects of antivirulence therapies. Nat. Rev. Micro. 6, 17-27.
15. CELIA AITKEN et al. “Nosocomial Spread of Viral Disease” Clin

Microbiol Rev. 2001 July; 14(3): 528-546
16. Charlson, E. S., Bittinger, K., Haas, A. R., Fitzgerald, A. S., Frank, I., Yadav, A., Bushman, F. D., and Collman, R. G. (2011). Topographical continuity of bacterial populations in the healthy human respiratory tract. Am. J. Respir. Crit. Care Med. 184, 957-963.
17. Chastre, J., Wolff, M., Fagon, J.-Y., Chevret, S., Thomas, F., Wermert, D., Clementi, E., Gonzalez, J., Jusserand, D., Asfar, P., et al. (2003). Comparison of 8 vs 15 days of antibiotic therapy for ventilator-associated pneumonia in adults: a randomized trial. JAMA 290, 2588-2598.
18. Chen, W., Jin, W., Hardegen, N., Lei, K.-J., Li, L., Marinos, N., McGrady, G., and Wahl, S. M., (2003). Conversion of peripheral CD4+CD25− naive T cells to CD4+CD25+ regulatory T cells by TGF-beta induction of transcription factor Foxp3. J. Exp. Med. 198, 1875-1886.
19. Chopin, M., Seillet, C., Chevrier, S., Wu, L., Wang, H., Morse, H. C., Belz, G. T., and Nutt, S. L. (2013). Langerhans cells are generated by two distinct PU.1-dependent transcriptional networks. J. Exp. Med. 210, 2967-2980.
20. Denney, L., Byrne, A. J., Shea, T. J., Buckley, J. S., Pease, J. E., Herledan, G. M. F., Walker, S. A., Gregory, L. G., and Lloyd, C. M. (2015). Pulmonary Epithelial Cell-Derived Cytokine TGF-β1 Is a Critical Cofactor for Enhanced Innate Lymphoid Cell Function. Immunity 43, 945-958.
21. Geissmann, F., Manz, M. G., Jung, S., Sieweke, M. H., Merad, M., and Ley, K. (2010). Development of monocytes, macrophages, and dendritic cells. Science 327, 656-661.
22. Guilliams, M., Ginhoux, F., Jakubzick, C., Naik, S. H., Onai, N., Schraml, B. U., Segura, E., Tussiwand, R., and Yona, S. (2014). Dendritic cells, monocytes and macrophages: a unified nomenclature based on ontogeny. Nat. Rev. Immunol. 14, 571-578.
23. Guilliams, M., Lambrecht, B. N. and Hammad, H. (2013). Division of labor between lung dendritic cells and macrophages in the defense against pulmonary infections. Mucosal Immunol. 6, 464-473.
24. Hambleton, S., Salem, S., Bustamante, J., Bigley, V., Boisson-Dupuis, S., Azevedo, J., Fortin, A., Haniffa, M., Ceron-Gutierrez, L., Bacon, C. M., et al. (2011). IRF8 mutations and human dendritic-cell immunodeficiency. N. Engl. J. Med. 365, 127-138.
25. Hemmi, H., Takeuchi, O., Kawai, T., Kaisho, T., Sato, S., Sanjo, H., Matsumoto, M., Hoshino, K., Wagner, H., Takeda, K., and Akira, S. (2000). A Toll-like receptor recognizes bacterial DNA. Nature 408, 740-745.
26. Hotchkiss, R. S., Monneret, G., and Payen, D. (2013a). Sepsis-induced immunosuppression: from cellular dysfunctions to immunotherapy. Nat. Rev. Immunol. 13, 862-874.
27. Hotchkiss, R. S., Monneret, G. and Payen, D. (2013b). Immunosuppression in sepsis: a novel understanding of the disorder and a new therapeutic approach. Lancet Infect. Dis. 13, 260-268.
28. Jackson, J. T., Hu, Y., Liu, R., Masson, F., D'Amico, A., Carotta, S., Xin, A., Camilleri, M. J., Mount, A. M., Kallies, A., et al. (2011). Id2 expression delineates differential checkpoints in the genetic program of CD8α+ and CD103+ dendritic cell lineages. EMBO J. 30, 2690-2704.
29. Jung, S., Unutmaz, D., Wong, P., Sano, G.-I., De los Santos, K., Sparwasser, T., Wu, S., Vuthoori, S., Ko, K., Zavala, F., et al. (2002). In vivo depletion of CD11c+ dendritic cells abrogates priming of CD8+ T cells by exogenous cell-associated antigens. Immunity 17, 211-220.
30. Kallies, A., Hasbold, J., Tarlinton, D. M., Dietrich, W., Corcoran, L. M., Hodgkin, P. D., and Nutt, S. L. (2004). Plasma cell ontogeny defined by quantitative changes in blimp-1 expression. J. Exp. Med. 200, 967-977.
31. Kamath, A. T., Henri, S., Battye, F., Tough, D. F., and Shortman, K. (2002). Developmental kinetics and lifespan of dendritic cells in mouse lymphoid organs. Blood 100, 1734-1741.
32. Kashiwagi, I., Morita, R., Schichita, T., Komai, K., Saeki, K., Matsumoto, M., Takeda, K., Nomura, M., Hayashi, A., Kanai, T., and Yoshimura, A. (2015). Smad2 and Smad3 Inversely Regulate TGF-β Autoinduction in Clostridium butyricum-Activated Dendritic Cells. Immunity 43, 65-79.
33. Kim, S. J., Zou, Y. R., Goldstein, J., Reizis, B., and Diamond, B. (2011). Tolerogenic function of Blimp-1 in dendritic cells. J. Exp. Med. 208, 2193-2199.
34. Köntgen, F., Suss, G., Stewart, C., Steinmetz, M., and Bluethmann, H. (1993). Targeted disruption of the MHC class II Aa gene in C57BL/6 mice. Int. Immunol. 5, 957-964.
35. Lahl, K., Loddenkemper, C., Drouin, C., Freyer, J., Arnason, J., Eberl, G., Hamann, A., Wagner, H., Huehn, J., and Sparwasser, T. (2007). Selective depletion of Foxp3+ regulatory T cells induces a scurfy-like disease. J. Exp. Med. 204, 57-63.
36. Lahoud, M. H., Ahmet, F., Kitsoulis, S., Wan, S. S., Vremec, D., Lee, C. N., Phipson, B., Shi, W., Smyth, G. K., Lew, A. M., et al. (2011). Targeting Antigen to Mouse Dendritic Cells via Clec9A Induces Potent CD4 T Cell Responses Biased toward a Follicular Helper Phenotype. J. Immunol. 187, 842-850.
37. Landmann, S., Mühlethaler-Mottet, A., Bernasconi, L., Suter, T., Waldburger, J. M., Masternak, K., Arrighi, J. F., Hauser, C., Fontana, A., and Reith, W. (2001). Maturation of dendritic cells is accompanied by rapid transcriptional silencing of class II transactivator (CIITA) expression. J. Exp. Med. 194, 379-391.
38. Leffleur et al. 2012 Production of human or humanized antibodies in mice. (2012). Production of human or humanized antibodies in mice., 901, 149-159
39. Marcel Geertz and Sebastian J. Maerkl, Experimental strategies for studying transcription factor—DNA binding specificities, Brief Funct Genomics. 2010 December; 9(5-6): 362-373
40. Marchingo, J. M., Kan, A., Sutherland, R. M., Duffy, K. R., Wellard, C. J., Belz, G. T., Lew, A. M., Dowling, M. R., Heinzel, S., and Hodgkin, P. D. (2014). T cell signaling. Antigen affinity, costimulation, and cytokine inputs sum linearly to amplify T cell expansion. Science 346, 1123-1127.
41. Mizgerd, J. P. (2006). Lung infection—a public health priority. PLoS Med. 3, e76.
42. Mohammed, J., Beura, L. K., Bobr, A., Astry, B., Chicoine, B., Kashem, S. W., Welty, N. E., IgyártÓ, B. Z., Wijeyesinghe, S., Thompson, E. A., et al. (2016). Stromal cells control the epithelial residence of DCs and memory T cells by regulated activation of TGF-β. Nat. Immunol. 17, 414-421.
43. Onishi, Y., Fehervari, Z., Yamaguchi, T., and Sakaguchi, S. (2008). Foxp3+ natural regulatory T cells preferentially form aggregates on dendritic cells in vitro and actively inhibit their maturation. Proc. Natl. Acad. Sci. USA 105, 10113-10118.
44. Ramalingam, R., Larmonier, C. B., Thurston, R. D., Midura-Kiela, M. T., Zheng, S. G., Ghishan, F. K., and Kiela, P. R. (2012). Dendritic cell-specific disruption of TGF-β receptor II leads to altered regulatory T cell phenotype and spontaneous multiorgan autoimmunity. J. Immunol. 189, 3878-3893.
45. Roberts, A. B., Sporn, M. B., Assoian, R. K., Smith, J. M., Roche, N. S., Wakefield, L. M., Heine, U. I., Liotta, L. A., Falanga, V., and Kehrl, J. H. (1986). Transforming growth factor type beta: rapid induction of fibrosis and angiogenesis in vivo and stimulation of collagen formation in vitro. Proc. Natl. Acad. Sci. USA 83, 4167-4171.
46. Roquilly, A., Braudeau, C., Cinotti, R., Dumonte, E., Motreul, R., Josien, R., and Asehnoune, K. (2013). Impaired blood dendritic cell numbers and functions after aneurysmal subarachnoid hemorrhage. PLoS ONE 8, e71639.
47. Roquilly, A., Feuillet, F., Seguin, P., Lasocki, S., Cinotti, R., Launey, Y., Thioliere, L., Le Floch, R., Mahe, P. J., Nesseler, N., et al. (2016). Empiric antimicrobial therapy for ventilator-associated pneumonia after brain injury. Eur. Respir. J. 47, 1219-1228.
48. Roquilly, A., Marret, E., Abraham, E., and Asehnoune, K. (2015). Pneumonia prevention to decrease mortality in intensive care unit: a systematic review and meta-analysis. Clin. Infect. Dis. 60, 64-75.
49. Roquilly, A., and Villadangos, J. A. (2015). The role of dendritic cell alterations in susceptibility to hospital-acquired infections during critical-illness related immunosuppression. Mol. Immunol. 68, 120-123.
50. Sabatel, C., Radermecker, C., Fievez, L., Paulissen, G., Chakarov, S., Fernandes, C., Olivier, S., Toussaint, M., Pirottin, D., Xiao, X., et al. (2017). Exposure to Bacterial CpG DNA Protects from Airway Allergic Inflammation by Expanding Regulatory Lung Interstitial Macrophages. Immunity 46, 457-473.
51. Sathe, P., Pooley, J., Vremec, D., Mintern, J., Jin, J.-O., Wu, L., Kwak, J.-Y., Villadangos, J. A., and Shortman, K. (2011). The acquisition of antigen cross-presentation function by newly formed dendritic cells. J. Immunol. 186, 5184-5192.
52. SCOTT K. FRIDKIN et al. “Epidemiology of Nosocomial Fungal Infection” Clin Microbiol Rev, 1996; 9(4): 499-511
53. Segura, E., and Villadangos, J. A. (2009). Antigen presentation by dendritic cells in vivo. Current Opin Immunol 21, 105-110.
54. Stary, G., Olive, A., Radovic-Moreno, A. F., Gondek, D., Alvarez, D., Basto, P. A., Perro, M., Vrbanac, V. D., Tager, A. M., Shi, J., et al. (2015). A mucosal vaccine against Chlamydia trachomatis generates two waves of protective memory T cells. Science 348, aaa8205-aaa8205.
55. Steinman, R. M., Hawiger, D., and Nussenzweig, M. C. (2003). Tolerogenic dendritic cells. Annu. Rev. Immunol. 21, 685-711.
56. Thompson, L. J., Lai, J.-F., Valladao, A. C., Thelen, T. D., Urry, Z. L., and Ziegler, S. F. (2016). Conditioning of naive CD4+ T cells for enhanced peripheral Foxp3 induction by nonspecific bystander inflammation. Nat. Immunol. 17, 297-303.
57. Travis, M. A., Reizis, B., Melton, A. C., Masteller, E., Tang, Q., Proctor, J. M., Wang, Y., Bernstein, X., Huang, X., Reichardt, L. F., et al. (2007). Loss of integrin alpha(v)beta8 on dendritic cells causes autoimmunity and colitis in mice. Nature 449, 361-365.
58. Trachtman, H., Fervenza, F. C., Gipson, D. S., Heering, P., Jayne, D. R. W., Peters, H., et al. (2011). A phase 1, single-dose study of fresolimumab, an anti-TGF-β antibody, in treatment-resistant primary focal segmental glomerulosclerosis. Kidney International, 79(11), 1236-1243
59. van Vught, L. A., Klein Klouwenberg, P. M. C., Spitoni, C., Scicluna, B. P., Wiewel, M. A., Horn, J., Schultz, M. J., Nurnberg, P., Bonten, M. J. M., Cremer, O. L., and van der Poll, T., MARS Consortium. (2016a). Incidence, Risk Factors, and Attributable Mortality of Secondary Infections in the Intensive Care Unit After Admission for Sepsis. JAMA 315, 1469-1479.
60. van Vught, L. A., Scicluna, B. P., Wiewel, M. A., Hoogendijk, A. J., Klein Klouwenberg, P. M. C., Franitza, M., Toliat, M. R., Nurnberg, P., Cremer, O. L., Horn, J., Schultz, M. J., Bonten, M. M. J., and van der Poll, T. (2016b). Comparative Analysis of the Host Response to Community-acquired and Hospital-acquired Pneumonia in Critically Ill Patients. Am. J. Respir. Crit. Care Med. 194, 1366-1374.
61. Vander Lugt, B., Khan, A. A., Hackney, J. A., Agrawal, S., Lesch, J., Zhou, M., Lee, W. P., Park, S., Xu, M., DeVoss, J., et al. (2013). Transcriptional programming of dendritic cells for enhanced MHC class II antigen presentation. Nat. Immunol. 15, 161-167.
62. Vega-Ramos, J., Roquilly, A., Zhan, Y., Young, L. J., Mintern, J. D., and Villadangos, J. A. (2014). Inflammation conditions mature dendritic cells to retain the capacity to present new antigens but with altered cytokine secretion function. J. Immunol. 193, 3851-3859.
63. Wakim, L. M., Gupta, N., Mintern, J. D., and Villadangos, J. A. (2013). Enhanced survival of lung tissue-resident memory CD8+ T cells during infection with influenza virus due to selective expression of IFITM3. Nat. Immunol. 14, 238-245.
64. Wakim, L. M., Smith, J., Caminschi, I., Lahoud, M. H., and Villadangos, J. A. (2015). Antibody-targeted vaccination to lung dendritic cells generates tissue-resident memory CD8 T cells that are highly protective against influenza virus infection. Mucosal Immunol. 8, 1060-1071.
65. Weiss, J. M., Bilate, A. M., Gobert, M., Ding, Y., Curotto de Lafaille, M. A., Parkhurst, C. N., Xiong, H., Dolpady, J., Frey, A. B., Ruocco, M. G., et al. (2012). Neuropilin 1 is expressed on thymus-derived natural regulatory T cells, but not mucosa-generated induced Foxp3+ T reg cells. J. Exp. Med. 209, 1723-1742.
66. Wilson, N. S., Behrens, G. M. N., Lundie, R. J., Smith, C. M., Waithman, J., Young, L., Forehan, S. P., Mount, A., Steptoe, R. J., Shortman, K. D., et al. (2006). Systemic activation of dendritic cells by Toll-like receptor ligands or malaria infection impairs cross-presentation and antiviral immunity. Nat. Immunol. 7, 165-172.
67. Worthington, J. J., Czajkowska, B. I., Melton, A. C., and Travis, M. A. (2011). Intestinal dendritic cells specialize to activate transforming growth factor-n and induce Foxp3+ regulatory T cells via integrin αvβ8. Gastroenterology 141, 1802-1812.

68. Vega-Ramos, J., Roquilly, A., Zhan, Y., Young, L. J., Mintern, J. D., & Villadangos, J. A. (2014). Inflammation conditions mature dendritic cells to retain the capacity to present new antigens but with altered cytokine secretion function. Journal of Immunology (Baltimore, Md.: 1950), 193(8), 3851-3859.
69. Ella Ott, Dr. med., et al. “The Prevalence of Nosocomial and Community Acquired Infections in a University Hospital An Observational Study Dtsch Arztebl Int. 2013 August; 110(31-32): 533-540
70. Gokhale et al. Single low dose rHuIL-12 safely triggers mutyilineage hematopoietic and immune-mediated effects, Experimental Hematology & oncology 2014, 3:11, pages 1-18

1. A method of preventing and/or treating a secondary infection, comprising administering to a subject in need thereof Interleukin 12 (IL12) or a derivative thereof.

2. (canceled)

3. The method of claim 1, wherein the secondary infection is selected from the group consisting of pneumonia, pleural infection, urinary infection, peritoneal infection, intra-abdominal abscess, meningitis, mediastinal infection, soft-tissue infection, and skin infection

4. The method of claim 1, wherein the interleukin 12 (IL12) or derivative thereof is administered in an amount from 2 to 20 μg.

5. The method of claim 1, wherein the interleukin 12 (IL12) or derivative thereof is administered in an amount from 0.1 μg/kg to 1 μg/kg.

6. The method of claim 1, wherein the interleukin 12 (IL12) or derivative thereof is administrated in a single injection or in repeated injections for up to 21 days.

7. (canceled)

8. A single use pharmaceutical composition comprising Interleukin 12 (IL 12) at a level from 2 to 20 μg per administration or 0.1 microg/kg to 1 microg/kg per administration.
